SEQUENCE LISTING (13 sequences)

SEQ ID NO: 1 - protein, 555 aa, Citrobacter freundii
MRRSKRFEVLAKRPVNQDGLIGEWPEEGLIAMESPYDPASSVKVENGRIVELDGKSRAEFDMIDRFIADYAINVAEAEHAMQLDALEIARMLVDIHVSREEIIAITTAITPAKAVEVMAQMNVVEMMMALQKMRARRTPSNQCHVTNLKDNPVQIAADAAEAGIRGFSEQETTVGIARYAPFNALALLVGSQCGRPGVLTQCSVEEATELELGMRGLTSYAETVSVYGTESVFTDGDDTPWSKAFLASAYASRGLKMRYTSGTGSEALMGYSESKSMLYLESRCIFITKGAGVQGLQNGAVSCIGMTGAVPSGIRAVLAENLIASMLDLEVASANDQTFSHSDIRRTARTLMQMLPGTDFIFSGYSAVPNYDNMFAGSNFDAEDFDDYNILQRDLMVDGGLRPVTEEETIAIRNKAARAIQAVFRELGLPLISDEEVEAATYAHGSKDMPARNVVEDLAAVEEMMKRNITGLDIVGALSCSGFEDIASNILNMLRQRVTGDYLQTSAILDRQFDVVSAVNDINDYQGPGTGYRISAERWAEIKNIAGVVQPGSIE

SEQ ID NO: 2 - protein, 194 aa, Citrobacter freundii
MECTTERKPVFTLQVSEGEAAKADDRADEVVIGVGPAFGKHQHKTLIDMPHNAILKELVAGIEEEGLHARVVRILRTSDVSFMAWDAANLSGSGIGIGIQSKGTTVIHQRDLLPLSNLELFSQAPLLTLETYRQIGKNAARYARKESPSPVPVVNDQMVRPKFMAKAALFHIKETKHVVQDAAPVTLHIALVRE

SEQ ID NO: 3 - protein, 144 aa, Citrobacter freundii
MTMNDNIMTAQDYPLATRCPEKILTPTGKPLTDITLENVLAGHVGPQDVRISQQTLEYQAQIAEQMQRHAVARNFRRAAELIAIPDTRILEIYNALRPFRSSFAELQAIADELEHTWHATVNAGFVRESAEVYQQRNKLRKGSQ

SEQ ID NO: 4 - protein, 603 aa, Citrobacter freundii
MPLIAGIDIGNATTEVALAQHGRFIASGIVATTGMKGTRDNIAGVVASLQQALEKTPWPLNDVAKICINEAAPVIGDVAMETITETIITESTMIGHNPQTPGGVGVGMGTTIAVEKLAALSEDRFAQGRIPLVGEEMDFLEAVWLINEALDRGINVVAAILKKDDGVLVNNRLRRTMPVVDEVTLLEKVPEGVLAAVEVAAAGQVVRALSNPYGIATFFALTPEETQAIVPIARALIGNRSAVVLKTPQGDVRSRVIPAGKIFIRGERRGDEADVAQGAQAIMQAMSACAPVCDIRGEAGTHAGGMLERVRKVMASLSGHDMNAVHIQDLLAVDTFIPRKVQGGIAGECSMENAVGIAAMVKSDRLQMQAIASELSARLNTAVEVGGVEANMAVTGALTTPGCAAPLAILDLGAGSTDAAIINSEGAVKAVHLAGAGNMVSLLIQTELGLSDPFLAEEIKKYPLAKVESLFSIRHENGVVEFFREPLSPSVFAKVVYLKDGELIPVDNQTSLEKIRLVRRQAKEKVFVTNCLRALRQVSPGGSIRDITFVVLVGGSSLDFEIPQMITDALAHYGVVAGQGNIRGTEGPRNAVATGLVLAGIAN

SEQ ID NO: 5 - protein, 117 aa, Citrobacter freundii
MSLSSPSVHLFYHPRWQDTRALDELCWGLEEQGVPCRTVCCDGNDCALALSKLAAKSSTLRVGLGLNATGDIALTHAQLPEDRALVGGHITAGMAQIRTLGANAGQLVKVLPFSEIT

SEQ ID NO: 6 - protein, 787 aa, Clostridium butyricum
MISKGFSTQTERINILKAQILNAKPCVESERAILITESFKQTEGQPAILRRALALKHILENIPITIRDQELIVGSLTKEPRSSQVFPEFSNKWLQDELDRLNKRTGDAFQISEESKEKLKDVFEYWNGKTTSELATSYMTEETREAVNCDVFTVGNYYYNGVGHVSVDYGKVLRVGFNGIINEAKEQLEKNRSIDPDFIKKEKFLNSVIISCEAAITYVNRYAKKAKEIADNTSDAKRKAELNEIAKICSKVSGEGAKSFYEACQLFWFIHAIINIESNGHSISPARFDQYMYPYYENDKNITDKFAQELIDCIWIKLNDINKVRDEISTKHFGGYPMYQNLIVGGQNSEGKDATNKVSYMALEAAVHVKLPQPSLSVRIWNKTPDEFLLRAAELTREGLGLPAYYNDEVIIPALVSRGLTLEDARDYGIIGCVEPQKPGKTEGWHDSAFFNLARIVELTINSGFDKNKQIGPKTQNFEEMKSFDEFMKAYKAQMEYFVKHMCCADNCIDIAHAERAPLPFLSSMVDNCIGKGKSLQDGGAEYNFSGPQGVGVANIGDSLVAVKKIVFDENKITPSELKKTLNNDFKNSEEIQALLKNAPKFGNDIDEVDNLAREGALVYCREVNKYTNPRGGNFQPGLYPSSINVYFGSLTGATPDGRKSGQPLADGVSPSRGCDVSGPTAACNSVSKLDHFIASNGTLFNQKFHPSALKGDNGLMNLSSLIRSYFDQKGFHVQFNVIDKKILLAAQKNPEKYQDLIVRVAGYSAQFISLDKSIQNDIIARTEHVM

SEQ ID NO: 7 - protein, 304 aa, Clostridium butyricum
MSKEIKGVLFNIQKFSLHDGPGIRTIVFFKGCSMSCLWCSNPESQDIKPQVMFNKNLCTKCGRCKSQCKSAAIDMNSEYRIDKSKCTECTKCVDNCLSGALVIEGRNYSVEDVIKELKKDSVQYRRSNGGITLSGGEVLLQPDFAVELLKECKSYGWHTAIETAMYVNSESVKKVIPYIDLAMIDIKSMNDEIHRKFTGVSNEIILQNIKLSDELAKEIIIRIPVIEGFNADLQSIGAIAQFSKSLTNLKRIDLLPYHNYGENKYQAIGREYSLKELKSPSKDKMERLKALVEIMGIPCTIGAE

SEQ ID NO: 8 - DNA, 30 bp, Artificial Sequence
  forward primer K1
gcgccatggtaagtaaaggatttagtaccc

SEQ ID NO: 9 - DNA, 28 bp, Artificial Sequence
  reverse primer K2
cgggatcctattactcagctccaattgt

SEQ ID NO: 10 - protein, 367 aa, Escherichia coli
MDRIIQSPGKYIQGADVINRLGEYLKPLAERWLVVGDKFVLGFAQSTVEKSFKDAGLVVEIAPFGGECSQNEIDRLRGIAETAQCGAILGIGGGKTLDTAKALAHFMGVPVAIAPTIASTDAPCSALSVIYTDEGEFDRYLLLPNNPNMVIVDTKIVAGAPARLLAAGIGDALATWFEARACSRSGATTMAGGKCTQAALALAELCYNTLLEEGEKAMLAAEQHVVTPALERVIEANTYLSGVGFESGGLAAAHAVHNGLTAIPDAHHYYHGEKVAFGTLTQLVLENAPVEEIETVAALSHAVGLPITLAQLDIKEDVPAKMRIVAEAACAEGETIHNMPGGATPDQVYAALLVADQYGQRFLQEWE

SEQ ID NO: 11 - protein, 356 aa, Escherichia coli
MKKLINDVQDVLDEQLAGLAKAHPSLTLHQDPVYVTRADAPVAGKVALLSGGGSGHEPMHCGYIGQGMLSGACPGEIFTSPTPDKIFECAMQVDGGEGVLLIIKNYTGDILNFETATELLHDSGVKVTTVVIDDDVAVKDSLYTAGRRGVANTVLIEKLVGAAAERGDSLDACAELGRKLNNQGHSIGIALGACTVPAAGKPSFTLADNEMEFGVGIHGEPGIDRRPFSSLDQTVDEMFDTLLVNGSYHRTLRFWDYQQGSWQEEQQTKQPLQSGDRVIALVNNLGATPLSELYGVYNRLTTRCQQAGLTIERNLIGAYCTSLDMTGFSITLLKVDDETLALWDAPVHTPALNWGK

SEQ ID NO: 12 - protein, 370 aa, Escherichia coli
MKTFSLQTRLYSGTGSLNILSRFTNRHIWIICDVFLAHSPLIDTLHQALPDSNRLSIFSEITPDPTIQTVVKGIAQMQTLRPDVVIGFGGGSALDAAKAIVWFGRQCGIEIETCVAIPTTSGTGSEVTSACVISDPEKGIKYPLFDNALYPDIAILDPSLVVSVPPAITANTGMDVLTHALEAYVSPRASDFTDALVEKAVQIVFQYLPTAVKKGDCLATRGKMHNASTLAGIAFSQAGLGLNHALAHQLGGQFHLPHGLANALLLTAVIRFNAGDPRAAKRYARLAKTCHLCPDNANDTASLNALIQHIEQLKTTCTLPTLANALKEKKAEWSIRIPDMVQAALADATLRTNPRAADASAIAELLEELL

SEQ ID NO: 13 - protein, 495 aa, Escherichia coli
MNFHHLAYWQDKALSLAIENRLFINGEYTAAAENETFETVDPVTQAPLAKIARGKSVDIDRAVSAARGVFERGDWSLSSPAKRKAVLNKLADLMEAHAEELALLETLDTGKPIRHSLRDDIPGAARAIRWYAEAIDKVYGEVATTSSHELAMIVREPVGVIAAIVPWNFPLLLTCWKLGPALAAGNSVVLKPSEKSPLSAIRLAGLAKEAGLPDGVLNVVTGFGHEAGQALSRHNDIDAIAFTGSTRTGKQLLKDAGESNMKRVWLEAGGKSANIVFADCPDLQKAASATAAGIFYNQGQVCIAGTRLLLEESIADEFLALLKQQAQNWQPGHPLDPATTMGTLIDSAHADSVHSFIREGESKGQLLLDGRNAELAAAIGPTIFVDVDPNASLSREEIFGPVLVVTRFTSEDQALQLANDSQYGLGAAVWTRDLSRAHRMSRRLKAGSVFVNNYNDGDMTVPFGGYKQSGNGRDKSLHALEKFTELKTIWISLEA

(19) United States
(12) Patent Application Publication
(10) Pub. No.: US 2023/0357806 A1
(43) Pub. Date: Nov. 9, 2023

(54) FERMENTATION PROCESS TO PRODUCE BIOACROLEIN AND BIOACRYLIC ACID

(71) Applicant: GENOMIUM, INC., Flagler Beach, FL (US)

(72) Inventor: CENAN OZMERAL, FLAGLER BEACH, FL (US)

(21) Appl. No.: 18/029,120

(22) PCT Filed: Sep. 30, 2021
(86) PCT No.: PCT/US2021/071652

§ 371 Date: Mar. 29, 2023

Related U.S. Application Data
(60) Provisional application No. 63/085,896, filed on Sep. 30, 2020.

(51) Int. Cl. C12P 7/24; C12N 9/88; C07C 51/235; B01D 3/00; B01D 3/36

(52) U.S. Cl. CPC C12P 7/24; C12N 9/88; C12Y 402/0103; C07C 51/235; B01D 3/002; B01D 3/36; B01D 3/009

(57) ABSTRACT

A method for producing bioacrolein using renewable glycerol as a feedstock as well as a method for producing bioacrylic acid using bioacrolein as a feedstock are provided in the present invention. Also provided in the present invention are recombinant microbial cells useful in producing 3-hydroxypropionaldhyde from glycerol, method of converting the 3-hydroxypropionaldhyde into bioacrolein and a process for recovering acrolein using a fractional distillation process.

### CROSS-REFERENCE TO RELATED APPLICATION

This application claims the benefit of U.S. Provisional Application Ser. No. 63/085,896, filed Sep. 30, 2020, the disclosure of which is hereby incorporated by reference in its entirety, including all figures, tables and amino acid or nucleic acid sequences.

### STATEMENT REGARDING SEQUENCE LISTING

The Sequence Listing for this application is labeled “Seq-List.txt” which was created on Sep. 29, 2021 and is 38 KB. The entire content of the sequence listing is incorporated herein by reference in its entirety.

### FIELD OF THE INVENTION

The present invention is in the field of manufacturing bioacrolein using a biological fermentation process using renewable glycerol feedstock and collecting bioacrolein from the fermentation medium using an in-situ product recovery process involving fractional distillation process. The bioacrolein produced in the present invention can be used in a variety of downstream applications including the use as a feedstock in manufacturing bioacrylic acid.

### BACKGROUND OF THE INVENTION

Acrylic acid, a α,β-unsaturated carboxylic acid, is an important commodity chemical. When reacted with an alcohol, it forms the corresponding ester. Acrylic acid and its esters readily combine with themselves or other monomers by reacting at their double bond to form homopolymers or copolymers useful in the manufacture of various plastics, coatings, adhesives, superabsorbents, elastomers, floor polishes and paints. Superabsorbents used in diapers, adult incontinence pads and feminine hygiene products are by far the largest use of acrylic acid and exhibits very strong growth (5.0-6.0% per annum).

Traditionally, acrylic acid is derived from fossil hydrocarbon resources. The most widely used process for acrylic acid is the vapor phase oxidation of propylene, which is a by-product of ethylene and gasoline production, involving two reactions in series, using two separate catalysts. As propylene passes through the first reactor, an intermediate product comprising acrolein and/or allyl alcohol is produced. When the intermediate product generated in the first reactor passes through the second reactor, it is oxidized to acrylic acid. The catalysts used in these two reactors are quite expensive and needs to be replaced every 3-4 years. Replacing the catalyst in a standard size reactor costs around $6-7 MM and requires decommissioning of the reactor for 3-4 weeks. Each reactor has 10,000 tubes filled with the solid catalyst. In the process of catalyst replacement, these reactor tubes are hydro-blasted to remove spent catalyst, inspected for damages and refilled with fresh catalyst. The other method for acrylic acid manufacturing involves hydroxycarboxylation of acetylene. This method utilizes nickel carbonyl and high-pressure carbon monoxide, both of which are expensive and considered environmentally unfriendly. In addition, there is a concern in the continued use of fossil hydrocarbon reserves in the manufacture of acrylic acid as it contributes to an increase in the greenhouse gas emission. As a result, there is a growing interest in using renewable organic carbon resources such as glucose, sucrose, fructose, glycerol and cellulosic hydrolysate as a feedstock in acrylic acid manufacturing.

Efforts have been made to produce acrylic acid and its esters through catalytic dehydration of lactic acid or 3-hydroxypropionic acid derived from renewable biological resources like sugar cane, corn and cellulosic feedstock. A number of inorganic solid acid catalysts have been reported to be useful in the production of acrylic acid from lactic acid at elevated temperature. The production of acrylic acid from lactic acid involves removal of hydroxyl group from alpha carbon atom and hydrogen atom from the adjacent beta carbon atom. Thus, it would appear that the efficiency of this chemical conversion from lactic acid to acrylic acid would depend on the rate constant for lactic acid dehydration reaction. But in reality, the challenge in increasing the efficiency of dehydration of lactic acid leading to acrylic acid production depends on inhibiting a number of competing side reactions which requires complex downstream processing and a lot of development work.

There has been a proposal to use 1, 3 propanediol derived from renewable biological resources such as glucose and glycerol in the manufacture of acrylic acid. Again, as is the case with use of biolactic acid as a feedstock in acrylic acid manufacturing, there have been a number challenges in developing commercial scale bio-1,3 propanediol based acrylic acid manufacturing. Thus, there is a need for developing a technology for manufacturing bioacrylic acid. The present invention proposes a novel, cost-effective technology to manufacture bioacrolein from renewable glycerol available as a by-product from the biodiesel industry. The bioacrolein manufactured according to the present invention can be used as a feedstock in manufacturing bioacrylic acid using the second reactor in the existing traditional acrylic acid plants.

Glycerol proposed as a feedstock in this present invention for bioacrolein manufacturing is derived from plant oils in the production of biodiesel fuel or oleochemicals such as fatty acids or fatty alcohol or fatty esters. Since glycerol is amenable to dehydration using chemical catalysts, it has been under consideration for commercial scale acrolein manufacturing. Glycerol is one of the raw materials envisaged as a substitute for propylene. Glycerol can be subjected to a catalytic dehydration reaction in order to produce acrolein. A large number of chemical catalysts have already been tested in the dehydration reaction of glycerol to acrolein. However, a chemical catalytic process for bioacrolein production using glycerol as a feedstock results in the production of undesirable side products and the removal of these side products using appropriate complex downstream processing adds cost making this chemical catalytic process not suitable for commercial scale manufacturing of bioacrylic acid using glycerol as a feedstock. For this reason, the chemical catalytic conversion process is yet to yield an acrolein stream free of by-products which can be used as a feedstock in the bioacrylic acid manufacturing.

The present invention discloses a novel, environmentally friendly process for manufacturing bioacrolein using glycerol as a feedstock. The bioacrolein manufactured according to the present invention can be fed into the second reactor of the existing commercial scale acrylic acid plants to produce bioacrylic acid without the need for any new capital expenses. Use of bioacrolein derived from the proposed process would help to overcome the problem in manufacturing the acrolein from petrochemical feedstock. In one embodiment, the present invention provides microbial catalyst for the production of 3-hydroxypropionaldhyde using glycerol as a feedstock. In another embodiment, the present invention provides a process of converting 3-hydroxypropanaldehyde into bioacrolein and recovering bioacrolein through an in-situ fractional distillation process. In yet another embodiment, the present invention provides a process for manufacturing bioacrylic acid using bioacrolein manufactured according to the present invention.

### SUMMARY OF THE INVENTION

The present invention relates to a process for preparing bioacrylic acid using bioacrolein as a feedstock. In one embodiment, the present invention provides a method for producing bioacrolein using bio-3-hydroxypropionaldehyde produced through microbial fermentation process using microbial biocatalysts and renewable glycerol as a feedstock.

In one aspect of the present invention, the microorganism used for the fermentative production of bio-3-hydroxyprpionaldehyde is isolated from natural environments through screening for bio-3-hydroxypropionaldehyde production using glycerol as a feedstock. In a preferred aspect of the present invention, natural microbial isolates selected for their ability to produce bio-3-hydroxypropionaldehyde using glycerol as a feedstock is subjected to further genetic manipulations to block all glycerol utilization pathways within the microbial cell other than the one which is required to produce bio-3-hydroxyprpionaldehyde from glycerol.

In another embodiment, the present invention provides the recombinant microorganisms expressing an exogenous gene coding a glycerol dehydratase responsible for the production of 3-hydroxypropinaldehyde using glycerol as a substrate. In one aspect, the exogenous glycerol dehydratase enzyme within the recombinant microorganism is dependent on B12 coenzyme for its function and such a glycerol dehydratase enzyme is referred herein as B12-dependendent glycerol dehydratase. In a preferred aspect of the invention, the recombinant microorganisms expressing a B12-dependent exogenous gene coding for glycerol dehydratase further comprises an exogenous gene coding for a protein functioning as an activator of inactivated B12-dependent glycerol dehydratase. In another aspect of the present invention, the exogenous glycerol dehydratase enzyme within the recombinant microorganism functions without the requirement for B12 coenzyme and such a glycerol dehydratase enzyme is referred herein as B12-independent glycerol dehydratase. In yet another aspect of the present invention, the recombinant microorganism expressing B12-dependent glycerol dehydratase further comprise genes coding for the enzymes responsible for the synthesis of B12 coenzyme. In a preferred aspect of the invention, the recombinant microorganisms expressing a B12-independent exogenous gene coding for glycerol dehydratase further comprises an exogenous gene coding for a protein functioning as an activator of inactivated B12-independent glycerol dehydratase. In yet another aspect of the present invention, the recombinant microorganism comprising either a B12-dependent glycerol dehydratase or a B12-independent glycerol dehydratase is subjected to further genetic manipulations to block all glycerol utilization pathways within the recombinant microbial cell other than the one which produces bio-3-hydroxyprpionaldehyde from glycerol. In another preferred aspect of the invention, the recombinant microorganism expressing exogenous gene coding for glycerol dehydratase is an acidophilic microorganism with the ability grow and metabolize glycerol in an acidic environment. In yet another preferred aspect of the invention, the recombinant microorganism expressing exogenous gene coding for glycerol dehydratase is a thermophile with the ability grow and metabolize glycerol at an elevated temperature.

In another embodiment, the present invention provides methods for genetically engineered B12-dependent glycerol dehydratase enzyme and B12-independent glycerol dehydratase enzymes that have low pH tolerance, high temperature tolerance and resistance to suicidal inactivation. In yet another embodiment, the present invention provides microbial biocatalysts with improved efficiency for glycerol uptake.

In water, 3-hydroxypropionaldehyde undergoes spontaneous dehydration reaction to yield acrolein. Around neutral pH, acrolein and 3-hydroxypropionladhyde are in equilibrium. Under acidic conditions, equilibrium between 3-hydroxypropionaldehyde and acrolein shifts towards acrolein. Similarly, an increase in the temperature shifts the equilibrium between 3-hydroxypropionladehyde and acrolein towards acrolein. As defined in this invention, chemical equilibrium between 3-hydroxypropionladehyde and acrolein does not mean that at equilibrium, 3-hydroxypropionladehyde and acrolein have the same molar concentrations. Instead, at a specific pH and temperature, the molar ratio of 3-hydroxypropionladehyde and acrolein remains constant in spite of the back and forth conversion of 3-hydroxypropionladehyde and acrolein. The fact that 3-hydroxypropionaldehyde and acrolein are in equilibrium just means that these molecules will migrate from one side of the equation to other side while the molar ratio between the molecules on both sides of the equation remains constant. Most likely, at neutral pH and normal temperature and pressure (20° C./68° F. and 1 atm) the molar ratio between 3-hydroxypropionaldehyde to acrolein is more than 1, 2, 3, 4, 5, 10 or 100. Under acidic conditions, the molar ratio between 3-hydroxypropionaldehyde to acrolein is less than 1, 0.9, 0.8, 0.6, 0.5, 0.1, 0.05, 0.01, 0.005 or 0.001. At elevated temperature, the molar ratio between 3-hydroxypropionaldehyde to acrolein is less than 1, 0.9, 0.8, 0.6, 0.5, 0.1, 0.05, 0.01, 0.005 or 0.001.

When 3-hydroxypropionaldehyde and acrolein are in an equilibrium at specified temperature and pH, a certain number of 3-hydroxypropionaldehyde molecules will lose a water molecule to become molecules of acrolein while at the same time, a similar number of acrolein molecule will acquire a molecule of water to become molecules of 3-hydroxypropionaldehyde. The molar concentration of 3-hydroxypropionaldehyde and acrolein can be determined following appropriate chemical assays.

One way to influence a chemical conversion under an equilibrium condition is to utilize Le Chatelier's principle. According to this chemical principle if a constraint (such as a change in pressure, temperature, or concentration of a reactant) is applied to a system in equilibrium, the equilibrium will shift so as to counteract the effect of the constraint. In the instant example, where 3-hydroxypropionladehyde and acrolein in an aqueous environment are in a chemical equilibrium, removal of acrolein from the aqueous medium would favor the dehydration of 3-hydroxypropionaldehyde to form more acrolein until the fixed original molar ratio between 3-hydroxypropionaldehyde and acrolein is achieved. If there is a continuous removal of acrolein through fractional distillation, there would be a continuous conversion of 3-hydroxypropionaldehyde to acrolein. As a way of an example, let us a assume in an aqueous solution, there is a chemical equilibrium between 3-hydroxypropionaldehyde and acrolein with a molar ratio of 4 (80 moles of 3-hydroxypropionaldehyde and 20 moles of acrolein) at 37° C. Since acrolein has a boiling point of 53° C., heating the aqueous solution containing 3-hydroxypropionaldehyde and acrolein to 53° C. would allow the evaporation of acrolein and thereby increase the molar ratio between 3-hydroxypropionaldehyde and acrolein in the aqueous phase which in turn would force the chemical equilibrium within the aqueous phase more towards the conversion of 3-hydroxypropionaldehyde to acrolein to regain the original molar ratio of 4. To begin with, if there were 80 moles of 3-hydroxypropionaldehyde and 20 moles of acrolein in the aqueous phase, the molar ratio between 3-hydroxypropionaldehyde and acrolein would be 4 (80:20). If raising of the temperature to acrolein's boiling point of 53° C., would remove 10 moles of acrolein from the aqueous phase, the molar ratio between 3-hydroxypropionaldehyde and acrolein would be expected to increase to 8 (80:10). Under the Le Chatelier's principal, 8 moles of 3-hydroxypropionaldehyde would be converted into acrolein for the purpose of regaining the original molar ratio of 4 between 3-hydroxypropionaldehyde and acrolein (72:18). If there is a continuous evaporation of acrolein from the aqueous phase, there would be continuous conversion of 3-hydroxypropionaldehyde to acrolein as soon as 3-hydroxypropionaldehyde accumulates in the aqueous phase.

Besides increasing the temperature of aqueous phase to 53° C. to allow the evaporation of acrolein, one could lower the vapor pressure to 50 to 100 mbar within the reaction vessel containing 3-hydroxypropionaldehyde and acrolein to lower the boiling point of acrolein from 53° C. to 37° C. so that the acrolein could evaporate from the aqueous phase at an ambient temperature as low as 37° C. forcing the continuous conversion of 3-hydroxypropionaldehye to acrolein.

Since acrolein has a lower boiling point (53° C.) when compared to the boiling point of 3-hydroxypropionaldehyde (175° C.) and water (100° C.), acrolein can be separated from 3-hydroxypropionladehyde using a fractional distillation process.

In one embodiment, the present invention provides a fractional distillation process for the recovery of bioacrolein from the fermentation broth comprising 3-hydroxypropionaldehyde and bioacrolein. In this fractional distillation process, the fermentation broth comprising 3-hydroxypropionaldehyde and bioacrolein is subjected to reduced pressure to induce the evaporation of acrolein at a temperature lower than 53° C. and the bioacrolein in the vapor phase is collected as a distillate. This in-situ bioacrolein recovery process coupled with a continuous fermentation process assures the efficiency of conversion of glycerol to bioacrolein besides overcoming the cytotoxic effects of -3-hydroxypropionaldehyde above certain concentration on the microbial biocatalyst used in the fermentation broth.

The process for bioacrolein production as described in this invention does not involve any costly purification step as bioacrolein is recovered in a pure form by using fractional distillation process at a low temperature of 53° C. At this temperature, the breakdown of biological molecules such as proteins and nucleic acids is kept at minimum. As a result, there is only a minimal amount of impurities associated with bioacrolein recovered using fractional distillation process according to the present invention. Moreover, by means of lowering the vapor pressure within the fermentation vessel, the temperature for fractional distillation of bioacrolein may further be lowered. In addition, due to the use of in-situ bioacrolein recovery process used in the present invention, water usage is also kept at minimum and thereby eliminating the need for recycling or disposing water stream that would result from a batch fermentation process.

In one embodiment, an acidophilic microorganism is used in the glycerol fermentation for the production of 3-hydroxypropinaldehyde. In one aspect of the present invention, the acidophilic microorganism used in the glycerol fermentation for the production of 3-hydroxypropinaldehyde contains an endogenous glycerol dehydratase gene. In a preferred aspect of this embodiment, the acidophilic microorganism used in the glycerol fermentation for the production of 3-hydroxypropionaldehyde is a recombinant microorganism comprising an exogenous gene coding either for a B12-dependent glycerol dehydratase or a B12-independent glycerol dehydratase and the glycerol fermentation is carried out at an acidic pH so that most of the 3-hydroxypropionaldehyde produced during glycerol fermentation is converted into bioacrolein enabling a higher yield for bioacrolein recovery in the downstream process involving fractional distillation.

In another embodiment of the present invention, a thermophilic microorganism is used in the glycerol fermentation for the production of 3-hydroxypropionaldehyde and the glycerol fermentation is carried out at an elevated temperature so that the need for a reduced vapor pressure required to lower the boiling point of bioacrolein in the fractional distillation process is overcome. In one aspect of the present invention, the thermophilic microorganism used in the glycerol fermentation for the production of 3-hydroxypropinaldehyde comprises an endogenous glycerol dehydratase gene. In a preferred aspect of this embodiment, the thermophilic microorganism used in the glycerol fermentation for the production of 3-hydroxypropionaldehyde is a recombinant microorganism comprising an exogenous gene coding either for a B12-dependent glycerol dehydratase or a B12-independent glycerol dehydratase and the glycerol fermentation is carried out at an elevated temperature.

In yet another embodiment, the present invention provides a process for producing bioacrylic acid using bioacrolein derived from distillation process. In one aspect of the present invention, bioacrolein is subjected to oxidation using chemical catalysts to produce bioacrylic acid. In another aspect of the present invention, bioacrylic acid is produced by subjecting bioacrolein to oxidation using chemical catalysts in the second reactor of a commercial scale acrylic acid plant currently using petrochemical feedstock.

The acrylic acid manufacturing process based on petrochemical feedstocks consists of two major steps. In the first reactor, propylene is subjected to catalytic oxidation to yield acrolein. In the second reactor, acrolein produced in the first reactor is oxidized to yield a very crude mixture of acrylic acid which is subjected to distillation process to remove some of the impurities to obtain crude acrylic acid mixture which is subjected to further distillation and crystallization process to obtain glacial acrylic acid.

The current industrial process for producing crude and purified glacial acrylic acid from propylene is a lengthy high-temperature process which introduce multitude of impurities including acetic acid, propionic acid, maleic acid and maleic anhydride, formaldehyde, furfural, benzaldehyde and acrylic acid oligomers. These impurities hinder polymerization of acrylic acid (for example in the production of superabsorbents), decrease polymerization degree and cause color formation. It is difficult to separate these impurities, especially furfural, due to the close boiling point to that of acrylic acid. For this reason, the crude acrylic acid is treated with chemicals such as amines and hydrazine in order to raise the boiling point of these impurities. Similarly, propionic acid is typical impurity in acrylic acid and it has boiling point as of acrylic acid (of 141° C.) and making it as a challenge remove propionic acid. Therefore, a process for preventing the formation of these impurities in the acrylic acid production is of great advantage in manufacturing acrylic acid in commercial scale.

In the industrial propylene-based acrylic acid manufacturing process, the impurities could reach 4-5% by weight of the finished product. During the distillation process these impurities buildup within the reactor as deposits on the column trays. Such deposits on the column trays makes the distillation process less efficient and makes it necessary to shut the plant once every month to remove the deposits. This is done mostly by entering the tower and water blasting the polymer buildup, which takes about 4-5 days. This is an expensive and potentially dangerous cleaning process and also reduces the plant's nameplate capacity by about 10-15%.

The bioacrolein production method according to the present invention using glycerol as feedstock eliminates all of the impurities associated with the acrolein manufactured using propylene as a feedstock. One exception is the formation of acrylic acid oligomers in the second reactor using acrolein. Since a highly pure form of bioacrolein is proposed as a feedstock for the second reactor, the acrylic acid oligomer formation in the second rector is expected to show a significant decrease. Thus, the process for manufacturing bioacrylic acid using glycerol as a feedstock is expected to have several desirable features over the current acrylic acid manufacturing process using propylene as a feedstock. This crude bioacrylic acid manufactured according to the present invention can be directly fed into the esterification units or, if desired, further purified to produce glacial bioacrylic acid.

In a traditional acrylic acid plant using propylene as a feedstock, the impurities that are accumulated as a result of extractive distillation are typically incinerated either on site or off site. Incineration of these chemicals is not only a significant expense but also known to result in dangerous (and regulated) gas emissions (NOxs). The process for manufacturing bioacrylic acid using glycerol as a feedstock according to the present invention will completely eliminate NOx emission associated with acrylic acid manufacturing.

Besides helping the nation to achieve its targets in developing its bio-economy, this invention is also attractive to chemical industry in achieving its sustainability goal. When it comes to bioacrylic acid manufacturing, current acrylic acid manufacturer stipulate that (1) production costs of bioacrylic acid must not exceed their current acrylic acid production cost based on petrochemical feedstock and (2) any proposed bioprocess technology for acrylic acid manufacturing should make use of their existing acrylic acid plants since billions of dollars have already been invested in building those plants. As explained in this patent application, the proposed process for the production of bioacrylic acid according to the present invention is cost effective. In addition, the bioacrolein produced in the first stage can be further processed using the second oxidation reactor and downstream isolation and purification equipment in the existing acrylic acid plants. For these reasons, the proposed invention will be attractive to chemical industry for investment and commercial scale manufacturing of bioacrylic acid.

### BRIEF DESCRIPTION OF THE DRAWINGS

The objects and features of the invention can be better understood with reference to the drawing described below, and the claims. The drawings are not necessarily to scale; emphasis instead generally being placed upon illustrating the principles of the invention. In the drawings and specification, like numerals are used to indicate like parts throughout the various views.

FIG. 1. Glycerol metabolism within a microbial cell. Shown in this FIGURE are three different glycerol metabolic pathways with in a microbial cell. Glycerol used in this invention is derived from renewable biological resources. In one metabolic pathway, glycerol is first converted into dihydroxyacetone by the action of NAD-linked glycerol dehydrogenase (1). Dihydroxyacetone is subsequently phosphorylated to produce dihydroxyacetone phosphate by the action of dihydroxyacetone kinase (2). Dihydroxyacetone phosphate enters into glycolytic pathway to produce energy and reducing power required for the normal growth and multiplication of the microbial cell. In the second glycerol metabolic pathways within a microbial cell, glycerol is converted into 3-hydroxypropionaldehyde by the action of the glycerol dehydratase enzyme (3). Subsequent hydrogenation of 3-hydroxypropionaldehyde by NADH-dependent oxidoreductase (4) yields 1, 3-propanediol. In another glycerol utilization pathway, by the action of aldehyde dehydrogenase (5), 3-hydroxypropionaldehyde is converted into 3-hydroxypropionic acid. In the microorganisms genetically engineered to produce 3-hydroxypropionaldehyde, the genes coding for the enzymes NAD-linked glycerol dehydrogenase (1), dihydroxyacetone kinase (2), NADH-dependent oxidoreductase (4) and aldehyde dehydrogenase (5) are mutated to block the pathway for the biosynthesis of dihydroxyacetone phosphate, 3-hydroxypropionic acid and 1, 3-propanediol. The blocked glycerol metabolic pathways are indicted by X in this FIGURE. 3-hydroxypropionladehyded resulting from the action of glycerol dehydratase enzyme on glycerol undergoes spontaneous dehydration reaction leading to the production of bioacrolein. Under neutral pH conditions within the microbial cells, bioacrolein and bio-3-hydroxypropionaldehyde are in an equilibrium and an acidic pH within the cell shifts this equilibrium towards bioacrolein accumulation. Bio-3-hydroxypropionaldehyde also tends to dimerize within the cell leading to the formation of reuterin, normally seen only at very high 3-hydroxypropionaldehyde concentrations.

### BRIEF DESCRIPTION OF THE SEQUENCES

SEQ ID NO: 1—Amino acid sequence of large subunit of glycerol dehydratase of Citrobacter freundii.

SEQ ID NO: 2—Amino acid sequence of middle subunit DhaC of glycerol dehydratase of Citrobacter freundii.

SEQ ID NO: 3—Amino acid sequence of small subunit DhaE of glycerol dehydratase of Citrobacter freundii.

SEQ ID NO: 4—Amino acid sequence of diol dehydratase reactivase subunit DhaF of Citrobacter freundii.

SEQ ID NO: 5—Amino acid sequence of diol dehydratase reactivase subunit DhaG of Citrobacter freundii.

SEQ ID NO: 6—Amino acid sequence of coenzyme B12-independent glycerol dehydratase DhaB1 subunit of Clostridium butyricum.

SEQ ID NO: 7—Amino acid sequence of coenzyme B12-independent glycerol dehydratase DhaB2 subunit of Clostridium butyricum.

SEQ ID NO: 8—Forward primer K1.

SEQ ID NO: 9—Reverse primer K2.

SEQ ID NO: 10—Amino acid sequence of NAD-linked glycerol dehydrogenase gldA of Escherichia coli.

SEQ ID NO: 11—Amino acid sequence of Dihydroxyacetone kinase subunit K (dhak) of Escherichia coli.

SEQ ID NO: 12—Amino acid sequence of NADH-dependent oxidoreductase (1,3-propanediol dehydrogenase, PPD) of Escherichia coli.

SEQ ID NO: 13—Amino acid sequence of Aldehyde dehydrogenase aldH of Escherichia coli.

### DETAILED DESCRIPTION

The present invention relates to a method of producing bioacrolein using renewable feedstocks using microbial cells as biocatalysts. More specifically, the present invention provides microbial biocatalysts that are useful in bioacrolein production by biological fermentation based on renewable feedstocks with very high yield, nearly 100% specificity and high titer for bioacrolein. Also provided in this invention is a process for recovering bioacrolein produced using the microbial biocatalysts of the present invention and subsequent conversion of bioacrolein to bioacrylic acid.

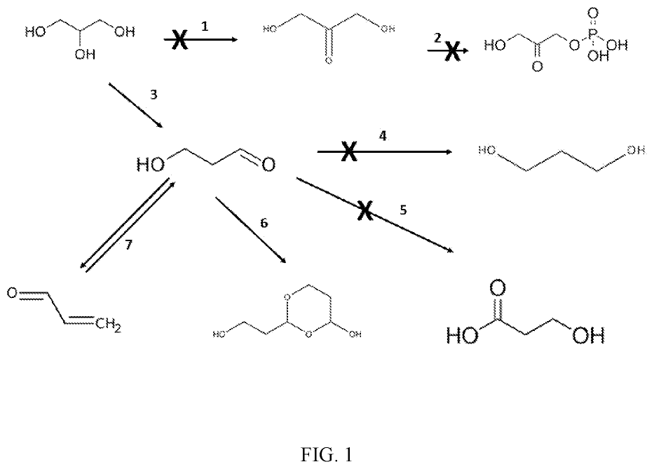

As used in the present invention, the term microbial biocatalyst refers to the microbial organisms useful in the production of desired chemicals including bioacrolein from renewable feedstocks through fermentation. Acrolein is the simplest unsaturated aldehyde (FIG. 1) and is also called propylene aldehyde, 2-propenal, 2-propen-1-one, prop-2-en-1-al, acrylaldehyde, acrylic aldehyde, allyl aldehyde, ethylene aldehyde and aqualine.

It should be noted that when “about” is used herein at the beginning of a numerical list, “about” modifies each number of the numerical list. It should be noted that in some numerical listings of ranges, some lower limits listed may be greater than some upper limits listed. One skilled in the art will recognize that the selected subset will require the selection of an upper limit in excess of the selected lower limit. The term “about” also provides for a range around a given value that is ±1%, 2%, 3%, 4%, 5%, 6%, 7%, 8%, 9%, or 10%. Thus, the term “about 10” includes a range of values that is between 9 and 11. Where the phrase “higher than about” is used, this phrase refers to a value that is 1%, 2%, 3%, 4%, 5%, 6%, 7%, 8%, 9%, or 10% higher than the state value. Where the phrase “lower than about” is used, the numerical value associated with that phrase is 1%, 2%, 3%, 4%, 5%, 6%, 7%, 8%, 9%, or 10% lower than the stated value.

As used herein, the term “yield” refers to the ratio of amount of product produced to the amount of feedstock consumed and it is usually expressed on a molar basis. For example, in the present invention, if 0.9 mole of bioacrolein is produced after consuming 1 mole of renewable glycerol, the yield for bioacrolein is 0.9 mole/mole.

As used herein, the term “titer” refers to amount of product produced per unit time and per unit volume of the fermentation fluid during the production phase of the fermentation process. For example, the titer for the bioacrolein production in the present invention can be expressed as gram of bioacrolein produced per liter of fermentation fluid per hour (g/1/hr.).

The term “selectivity” as used in the present invention refers to the percentage of a particular product formed in a chemical or biological reaction among the plurality of the products formed in that particular chemical or biological reaction. When a chemical or a biological reaction yields products “A”, “B” and “C”, the selectivity of that chemical reaction to the product “A” is obtained using the Equation: Moles of compound “A” formed/Moles of compounds “A”, “B” and “C” formed)×100. For example, if 100 mole of substrate is consumed to yield 50 moles of product A, 30 moles of product of B and 20 moles of product C, the specificity for products A, B and C is considered to be 50%, 30% and 20%, respectively. In the event, the product A is the only product derived from the substrate without any other products, the specificity for product A is said to be 100%. If product A is the only desired product, and products B and C are unwanted products, the product B and C are referred as side products or by-products. Under such a circumstance, in determining the specificity of the desired product A, the amount of side products B and C is taken into consideration.

As used herein, the term “renewable feedstock” refers to materials derived from plant biomass such as glucose, sucrose, glycerol and cellulosic hydrolysate. In the present invention the term renewable feedstock refers to glycerol derived as a by-product in the bio-diesel and other industries. A renewable feedstock is easily distinguishable from petrochemical feedstock by its ¹⁴C carbon content. In preferred embodiment of the present invention, the glycerol obtained as a byproduct in the biodiesel industry is used as feedstock in the manufacture of the bioacrolein.

The bioacrolein and bioacrylic acid manufactured according to the present invention can be distinguished from the acrolein and acrylic acid manufactured following the traditional methods involving petroleum feedstock on the basis of their ¹⁴C carbon content following the method ASTM-D6866 provided by American Society of Testing and Materials. Cosmic radiation produces ¹⁴C (“radiocarbon”) in the stratosphere by neutron bombardment of nitrogen. ¹⁴C atoms combine with oxygen atom in the atmosphere to form heavy ¹⁴CO₂, which, except in the radioactive decay, is indistinguishable from the ordinary carbon dioxide CO₂ concentration and the ¹⁴C/¹²C ratio is homogeneous over the globe and because it is used by the plants, the ratio ¹⁴C/¹²C is retained by the biomass while the content of ¹⁴C in the fossil materials, originally derived from photosynthetic energy conversion, has decayed due to its short half-life of 5730 years. By means of analyzing the ratio of ¹⁴C to ¹²C, it is possible to determine ratio of fossil fuel derived carbon to biomass-derived carbon International Patent Application Publication No. WO 2009/155085 A2 and U.S. Pat. No. 6,428,767 provide details about the use of ASTM-D6866 method for determining percent of biomass-derived carbon content in a chemical composition. International Patent Application Publication No. WO 2009/155085 A2 provides isocyanate and polyisocyanate compositions comprising more than 10 percent of carbon derived from renewable biomass resources. U.S. Pat. No. 6,428,767 provides a new polypropylene terephthalate composition. This new polypropylene terephthalate is comprised of 1,3-propanediol and terephthalate. The 1,3-propanediol used in this composition is produced by the bioconversion of a fermentable carbon source, preferably glucose. The resulting polypropylene terephthalate is distinguished from a similar polymer produced using petrochemical feedstock on the basis of dual carbon-isotopic fingerprinting which indicates the source and the age of the carbon. The details related carbon dating disclosed in the U.S. Pat. No. 6,428,767 is incorporated herein by reference. An application note from Perkin Elmer entitled “Differentiation Between Fossil and Biofuels by Liquid Scintillation Beta Spectrometry-Direct Method” provides details about the methods involving ASTM Standard D6866.

As used herein, the prefix “bio” in front of a chemical entity indicates that particular chemical entity is derived from a renewable feedstock which in turn is derived from renewable materials that are produced naturally in plants. As used herein, the term “plant biomass” includes any part of a plant biomass from which the renewable feedstocks such as glucose, fructose, sucrose glycerol and cellulosic hydrolysate can be derived. For example, triglycerides used as feedstock in the biodiesel industry are derived from one or other plant seeds and yield renewable glycerol upon hydrolysis.

As used herein, the term “polypeptide” comprises a particular amino acid sequence showing substantial identity to corresponding amino acid sequence. The term “substantial identity” means that one particular amino acid sequence shows at least 80%, preferably at least 90% homology when aligned with another test amino acid sequence and analyzed using algorithm commonly used in the art. For example, the polypeptide includes polypeptide that have an amino acid sequence having about 90% or more, 91% or more, 92% or more, 93% or more, 94% or more, 95% or more, 96% or more, 97% or more, 98% or more, 99% or more, 99.1% or more, 99.2% or more, 99.3% or more, 99.4% or more, 99.5% or more, 99.6% or more, 99.7% or more, 99.8% or more, or 99.9% or more identity to specific amino acid sequence, and is involved in the biosynthesis of 3-hydroxypropionaldehyde from glycerol. In general, as the identity percent of a particular polypeptide is higher, it is more preferable.

The list of the polypeptides having identity with a test amino acid sequence includes polypeptides that comprises an amino acid sequence having the deletion, substitution, insertion, and/or addition of an amino acid residue in the polypeptide of specific amino acid sequence. In general, as the number of deletion, substitution, insertion, and/or addition in a particular polypeptide is minimal, it is more preferable.

As used herein, the term “polynucleotide” includes DNA (gDNA and cDNA) and RNA molecules comprising nucleotides, which are the basic unit of a nucleic acid molecule. The term “nucleotide” as used herein includes sugar or base-modified analogues, as well as natural nucleotide. The polynucleotide of the present invention is not limited to a nucleic acid molecule encoding specific amino acid sequence (polypeptide), but also includes nucleic acid molecules encoding amino acid sequences showing substantial identity to the amino acid sequence or polypeptide having a function corresponding thereto. The term “a polypeptide having corresponding function” means that a particular polypeptide carries out its function just as the test polypeptide although it includes a deletion, a substitution, an insertion, and/or addition of at least one amino acid residue. Such polypeptides include polypeptides that consist of an amino acid sequence having the deletion, substitution, insertion, and/or addition of at least one amino acid residue, and are involved in the synthesis of bio-3-hydroxypropionaldehyde from renewable glycerol.

The identity between amino acid sequences or nucleotide sequences may be measured using the BLAST algorithm by Karlin and Altschul, according to BLASTN and BLASTX programs based on a BLAST algorithm. When a nucleotide sequence is analyzed using BLASTN, the parameters of, for example, score=100 and word length=12 can be used. If an amino acid sequence is analyzed using BLASTX, the parameters of, for example, score=50 and word length=3 can be used. In case BLAST and Gapped BLAST programs are used, default parameters are applied for each program.

As used herein, the term “expression cassette” refers to a portion of plasmid vector that comprises a promoter sequence, a sequence that codes for a gene of interest and a sequence that terminates transcription. When a microbial cell is transformed with a plasmid comprising an expression cassette, it is possible to integrate the expression cassette into the host chromosome. Such an integration of the expression cassette into the host chromosome is facilitated when the host chromosomal DNA sequences are present as flanking sequences on either side of the expression cassette in the plasmid vector.

As used herein the phrase “The transcription promoter” refers to a DNA sequence controlling the expression of coding sequence of gene of interest, including enhancers. The promoter may be a native promoter of the gene of interest or a heterogeneous promoter derived from another gene.

As used herein the phrase “transcription terminator sequence” refers to the nucleic acid sequence immediately downstream of gene of interest and is responsible for the termination of the transcription of the gene of interest.

As used herein, the term “dehydroxylation” refers to the removal of water from a reactant. The term “dehydroxylation” is also known as “dehydration” in the art.

The simplest pathway for bioacrolein synthesis within a microorganism starts with renewable glycerol as the substrate and involves only two steps. The first step of the acrolein synthetic pathway involves glycerol dehydratase enzyme which removes a water molecule from a molecule of glycerol to yield 3-hydroxypropionaldehyde (OH—CH2-CH2-CHO) as a product. In the second step of the bioacrolein synthetic pathway, 3-hydroxypropionaldehyde undergoes a spontaneous dehydration reaction to yield bioacrolein with a double bond between C2 and C3 carbon (H2C═CH—CHO). In water, acrolein and 3-hydroxypropionaldehyde are in equilibrium.

3-hydroxypropionaldehyde is found to be toxic to microbial cells at low concentration. As a result, the glycerol fermentation for the production of 3-hydroxypropionaldehyde is not sustainable for longer time unless 3-hydroxypropionaldehyde is removed from the fermentation broth before it reaches a critical concentration. One approach that has been followed to overcome the toxicity of 3-hydroxypropionaldehyde is to extract the 3-hydroxypropionaldehyde from the production medium using adsorbents such as semicarbazide-functionalized resins, chitosan polymers, hydrazides, hydrazines, hydrogen sulfites, sulfites, metabisulfites or pyrosulfites and the like as it is produced or before it reaches toxic levels. However, these methods are inefficient, costly and not scalable. These disadvantages do not allow for economic production and extraction of 3-hydroxypropionaldehyde on a larger scale.

Lactobacillus reuteri, has been shown to sustain large amounts of 3-hydroxypropionaldehyde production from the fermentation of glycerol. However, although L. reuteri is very resistant to high concentrations of 3-hydroxypropionaldehyde, its viability does decrease when 3-hydroxypropionaldehyde is produced in large quantities. Therefore, there exists a need for an in-situ process for recovering 3-hydroxypropionaldehye as it starts accumulating in the fermentation broth.

The present invention provides an in-situ fractional distillation process for removing 3-hydroxypropionaldehyde from the fermentation broth as soon as it is formed. 3-hydroxypropionaldehyde undergoes a spontaneous dehydration reaction leading to the formation of acrolein. The in-situ fractional distillation process according to the present invention is based on the fact that the acrolein has a much lower boiling point when compared to the boiling point of water and 3-hydroxypropionaldehyde and it is possible to separate acrolein using fractional distillation as soon as it is derived from 3-hydroxypropionaldehyde.

3-hydroxypropionaldehyde has a boiling point of 175° C. while acrolein has a boiling point of 53° C. Water has a boiling point of 100° C. By reducing the vapor pressure of the fermentation vessel used for producing 3-hydroxypropionaldehyde, the boiling point of acrolein can be further reduced as low as 37° C. Lowering the temperature for distillation process is expected to substantially reduce the energy requirement for recovering acrolein. In water, acrolein and 3-hydroxypropionaldehyde are in equilibrium and under acidic conditions this equilibrium shifts towards acrolein. As a result, by means of lowering the pH of the fermentation broth, the relative proportion of bioacrolein in the fermentation broth used in the reactive distillation process can be significantly increased. In addition, an increase in the temperature would shift the equilibrium between acrolein and 3-hydroxypropionaldehyde towards acrolein. Thus, by means of manipulating the fermentation conditions like acidity and temperature, it is possible to achieve maximum bioacrolein yield which is closer to the theoretical bioacrolein yield in the glycerol fermentation process accompanied by a distillation process. Once the bioacrolein is collected as a distillate, it is water free and it cannot go back to 3-hydroxypropionaldehyde. However, the purified bioacrolein obtained at the end of the reactive distillation process can be easily hydrated to obtain pure 3-hydroxypropionaldehyde when it is needed for certain applications.

One advantage of the distillation process according to the present invention is that the production, recovery, purification, and concentration of bioacrolein are carried out in one step. As a result, this process is well-suited for scale-up at minimal cost. Another advantage of the distillation process of the present invention is that there is no need for water separation when bioacrolein is used in chemical applications as the dehydration step is already integrated in the acrolein recovery process. Another important point to note here is that no harmful additives are used in the initial acrolein recovery process. As a result, in the bioacrolein recovery process according to the present invention, no waste stream is generated demanding additional cost involved in the disposal of waste stream.

Another advantage of bioacrolein production according to the present invention, is very high specificity for bioacrolein production from glycerol. The term specificity as used in the present invention refers to the relative percentage of bioacrolein produced when compared to the other bye-products formed in the process. Since bioacrolein is the only product derived from the spontaneous dehydration of 3-hydroxypropionladehyde, the specificity for bioacrolein production from 3-hydroxypropionaldehyde is expected to be closer to theoretical maximum of 100%, provided, that the dimerization of 3-hydroxypropionladehyde to reuterin is significantly reduced or completely eliminated under reduced vapor pressure or a slightly acidic condition used for reactive distillation processes. In addition, the microbial biocatalysts used in the present invention are genetically engineered to block all glycerol utilization pathways other than the conversion of glycerol to 3-hydroxypropionaldehyde. As a result, the yield and specificity for bioacrolein production from glycerol is expected to be closer to theoretical maximum, provided, that bioacrolein resulting from spontaneous dehydration of 3-hydroxypropionaldehyde is continuously removed through fractional distillation.

It should be realized at this point that to begin with, there is a limitation in realizing the optimal 3-hydroxypropionaldehyde yield in the fermentation process. Both 3-hydroxypropionaldehyde and bioacrolein are reported to have certain antibacterial properties. As soon as 3-hydroxypropionaldehyde is produced within the microbial cell, 3-hydroxypropionaldehyde and bioacrolein quickly accumulate to the level that is toxic to the host microbial cell. As a result, there is an advantage in the proposed fermentation process to remove bioacrolein as soon as it starts accumulating in the fermentation broth to assure the continuous 3-hydroxypropionaldehyde synthesis to obtain higher titer and yield for bioacrolein, the ultimate end product. The present invention provides the method for carrying out the fermentation at lower pH, at an elevated temperature and/or under reduced vapor pressure so that the reactive distillation can be initiated at the beginning of the fermentation instead of waiting till the end of the 3-hydroxypropinonaldehyde production phase in the fermentation process. This in-situ bioacrolein recovery process using fractional distillation allows the microbial cells to escape the toxic effect resulting from the accumulation of 3-hydroxypropionaldehyde and bioacrolein and assures the glycerol fermentation to 3-hydroxypropionaldehyde lasts for a longer duration.

To date, six genera of bacteria that are able to ferment glycerol into 3-hydroxypropionaldehyde have been identified: Bacillus (Voisenet 1914); Klebsiella (Aerobacter) (Abeles et al. 1960; Reymolds et al. 1939; Slininger et al. 1983); Citrobacter (Mickelson and Werkman 1940); Enterobacter (Barbirato et al. 1996); Clostridium (Humphreys 1924); and Lactobacillus (Mills et al. 1954; Serjak et al. 1954).

Certain strains of Lactobacillus reuteri are known to produce 3-hydroxypropionaldehyde. When 3-hydroxypropionaldehyde is excreted into the growth medium, it is found to have antimicrobial activities against Gram positive and Gram-negative bacteria, as well as, yeast, molds and protozoa. The antimicrobial agent based on 3-hydroxypropionaldehyde has come to be known as reuterin. A recent study, based on the finding that acrolein contributes to the antimicrobial and heterocyclic amine transformation activities of reuterin, has made a proposal to redefine reuterin to include acrolein. In vivo, active reuterin synthesis by Lactobacillus reutri could occur in the colon if sufficient amounts of glycerol become available as a product of luminal microbial fermentations. For this reason, L. reuteri has been used as a probiotic in human applications and is generally recognized as safe (GRAS) microorganism.

There has been a growing interest in producing reuterin for its use in the antimicrobial applications using L. reuteri in glycerol-based fermentation. The bioacrolein recovered from fermentation broth through distillation process according to the present invention can be used directly as an antimicrobial agent or alternatively be subjected to hydration reaction to yield 3-hydroxypropionaldehyde which in turn can be used in antimicrobial applications.

L. reuteri has been used for the production 1, 3 propanediol as well as 3-hydroxypropionicacid using glycerol as a feedstock. The production of 1, 3 propanediol using glycerol involves two different enzymes. In the first step of biosynthetic pathway for 1, 3 propanediol production, glycerol is converted to 3-hydroxypropionaldehyde by a vitamin B12-dependent glycerol dehydratase (GDH: EC 4.2.1.30) and in the second step, 3-hydroxypropionaldehyde is hydrogenated by NADH-linked oxidoreductase (PDOR: EC 1.1.1.202) to yield 1, 3 propanediol. As GDH and PDOR are most frequently co-expressed, 1, 3 propanediol constitutes a more easily accessible glycerol derivative than 3-hydroxypropionaldehyde. Glycerol conversion to 1, 3-propanediol enables the cells to replenish its NAD⁺ used during glycolysis. As a result, when the fermentation broth used to grow L. reuteri includes both glycerol and glucose, 1, 3 propanediol production from glycerol is a favored glycerol utilization pathway. When L. reuteri is grown in a medium containing only glycerol, the 1, 3 propanediol production from glycerol is going to be rate-limited for the lack of NADH. However, when L. reuteri is used as a microbial catalyst for 3-hydroxypropionaldehyde, it is desirable to inactivate the gene coding for NADH-linked oxidoreductase enzyme to block the conversion of 3-hydroxypropionaldehyde to 1, 3 propanediol.

Within the microbial cells, glycerol can also be converted into dihydroxyacetone, dihydroxy acetone phosphate and glyceraldehyde. Oxidation of glycerol, catalyzed by NAD-linked glycerol dehydrogenase, results in dihydroxyacetone. Dihydroxyacetone is phosphorylated by dihydroxyacetone kinase to yield dihydroxyacetone phosphate which is then funneled into the glycolytic pathway. Due to lack of dihydroxyacetone kinase enzyme, L. reuteri uses glycerol only for reductive conversion and hence needs an additional substrate for growth and energy production. In other words, the glycerol available to L. reuteri cannot be metabolized through glycolytic cycle to generate NADH. As a result, to produce 1,3-propanediol using glycerol as a feedstock with L. reuteri, there is a need to supply additional carbon source such as glucose which can be metabolized through glycolytic cycle and generate NADH required for the reduction of 3-hydroxypropionaldehyde to 1,3-propanediol.

In constructing a microbial strain for the production of 3-hydroxypropionaldehyde, in one embodiment, the pathway for the conversion of 3-hydroxypropionaldehyde to 1, 3-propanediol is blocked either by providing only glycerol as a source of carbon or by inactivating the NADH-dependent oxidoreductase responsible for the conversion of 3-hydroxypropionaldehyde to 1, 3-propanediol. In a preferred embodiment, the NADH-dependent oxidoreductase is inactivated by mutating the corresponding gene and the fermentation is carried out in two steps. In the first step, the microbial catalyst lacking functional NADH-dependent oxidoreductase is grown in a medium containing glucose as a carbon source. Once appropriate cell mass is accumulated and glucose in the medium is exhausted, glycerol is fed to the medium to induce the production of 3-hydroxypropionaldehyde.

In another embodiment of the present invention, as a way of directing the flow of carbon in the glycerol to the production of 3-hydroxypropionadehyde, the flow of carbon to dihydroxyacetone phosphate is blocked by means of mutating the genes encoding for NAD-linked glycerol dehydrogenase and dihydroxyacetone kinase enzymes. In another aspect of the present invention, the genes encoding for the NADH-dependent oxidoreductase, NAD-linked glycerol dehydrogenase and dihydroxyacetone kinase enzymes are mutated (FIG. 1).

As shown in FIG. 1, 3-hydroxypropinaldehyde resulting from the action of glycerol dehydratase acts as a substrate for the aldehyde dehydrogenase enzyme leading to the production of 3-hydroxypropionic acid. In the construction of a microbial catalyst for producing 3-hydroxypropionaldehyde from glycerol, it is necessary to block the path way for the conversion of 3-hydroxypropionaldehyde to 3-hydroxypropionic acid. In an ideal biocatalyst for the production of 3-hydroxypropinaldehyde using glycerol fermentation, it is necessary to inactivate the functioning of NADH-dependent oxidoreductase, NAD-linked glycerol dehydrogenase, dihydroxyacetone kinase and aldehyde dehydrogenase enzymes.

In another embodiment of the present invention, recombinant technology is used to construct a microbial catalyst that originally does not have glycerol dehydratase enzyme or the endogenous glycerol dehydratase enzyme is not efficient in 3-hyrdoxypropionaldehyde production using glycerol as a substrate. In one aspect of the present invention, an exogenous B12-dependent glycerol dehydratase enzyme is used in the construction of the recombinant microbial biocatalyst for 3-hyrdoxypropionaldehyde production. In another aspect of the present invention, an exogenous B12-independent glycerol dehydratase enzyme is used in the construction of the recombinant microbial biocatalyst for 3-hyrdoxypropionaldehyde production. In yet another aspect of the present invention, the recombinant microorganisms having exogenous genes wither for B12-dependent glycerol dehydratase enzyme or B12-independent glycerol dehydratase enzyme have the genes coding for the corresponding activation factor. In a preferred aspect of the present invention, besides using an exogenous B12-independent glycerol dehydratase enzyme in the construction of the recombinant microbial biocatalyst for 3-hyrdoxypropionaldehyde production, various glycerol utilization pathways other than the 3-hydroxypropanaldehyde pathway that exist within the acidophilic microorganism are blocked through appropriate genetic modifications.

The recombinant cell used for the production of 3-hyrdoxypropionaldehyde can be selected from the group consisting of Abiotrophia, Acaryochloris, Accumulibacter, Acetivibrio, Acetobacter, Acetohaloblum, Acetonema, Achromobacter, Acidaminococcus, Acidimicroblum, Acidiphillum, Acidithiobacillus, Acidobacterium, Acidothermus, Acidovorax, Acinetobacter, Actinobacillus, Actinomyces, Actinosynmema, Aerococcus, Aeromicrobium, Aeromonas, Afipla, Aggregatibacter, Agrobacterium, Ahrensia, Akkermansla, Alcanivorax, Alicycliphilus, Alicyclobaclllus, Aliivibrio, Alkalilimnicola, Alkaliphilus, Allochromatium, Alteromonadales, Alteromonas, Aminobacterlum, Aminomonas, Ammonmfex, Amycolatopsis, Amycolicicoccus, Anabaena, Anaerobaculum, Anaerococcus, Anaerofustis, Anaerolinea, Anaeromyxobacter, Anaerostipes, Anaerotruncus, Anaplasma, Anoxybacillus, Aqulfex, Arcanobacterlum, Arcobacter, Aromatoleum, Arthrobacter, Arthrospira, Asticcacaulis, Atopobium, Aurantimonas, Azoarcus, Azorhizobium, Azospirillum, Azotobacter, Bacillus, Bartonella, Basfia, Baumannia, Bdellovibrio, Beggiatoa, Bejerinckla, Bermanella, Beutenbergia, Bilidobacterium, Blophila, Blastopirellula, Blautia, Blochmannia, Bordetella, Borrella, Brachybacterlum, Brachyspira, Bradyrhizobium, Brevibacillus, Brevibacterium, Brevundimonas, Brucella, Buchnera, Bulleidia, Burkholdeia, Butyrivibrio, Caldalkalibacillus, Caldanaerobacter, Caldicellulosiruptor, Calditerrivibrio, Caminibacter, Campylobacter, Carboxydibrachlum, Carboxydothermus, Cardiobacterium, Camobacterium, Carsonella, Catenibacterium, Catenulispora, Catonella, Caulobacter, Cellulomonas, Cellvibrio, Centipeda, Chelativorans, Chloroflexus, Chromobacterium, Chromohalobacter, Chthoniobacter, Citrelcella, Citrobacter, Citromicrobium, Clavibacter, Cloacamonas, Clostridium, Collinsella, Colwellia, Comamonas, Conexibacter, Congregibacter, Coprobacillus, Coprococcus, Coprothermobacter, Coraliomargarita, Coriobacterlum, corrodens, Corynebacterium, Coxiella, Crocosphaera, Cronobacter, Cryptobacterium, Cupriavidus, Cyanobium, Cyanothece, Cylindrospermopsis, Dechloromonas, Defenibacter, Dehalococcoides, Dehalogenimonas, Deinococcus, Deiftia, Denitrovibrio, Dermacoccus, Desmospora, Desulfarculus, Desulphateibacillum, Desulfitobacterium, Desulfobacca, Desulfobacterium, Desulfobulbus, Desulfococcus, Desulfohalobium, Desulfomicrobium, Desulfonatronospira, Desulforu dis, Desulfotalea, Desulfotomaculum, Desulfovibrio, Desulfurlspirillum, Desulfurobacterlum, Desuluromonas, Dethiobacter, Dethiosulfovibrio, Dialister, Dichelobacter, Dickeya, Dictyoglomus, Dietzia, Dinoroseobacter, Dorea, Edwardsiella, Ehrlichia, Eikenella, Elusimicrobium, Endoriftia, Enhydrobacter, Enterobacter, Enterococcus, Epulopiscium, Erwinia, Erysipelothrix, Erythrobacter, Escherichia, Ethanoligenens, Eubacterium, Eubacterium, Exiguobacterium, Faecalibacterium, Ferrimonas, Fervidobacterium, Fibrobacter, Finegoldia, Flexistipes, Francisella, Frankia, Fructobacillus, Fulvimarina, Fusobacterium, Gallibacterium, Gallionella, Gardnerella, Gemella, Gemmate, Gemmatimonas, Geobacillus, Geobacter, Geodermatophilus, Glaciecola, Gloeobacter, Glossina, Gluconacetobacter, Gordonia, Granulibacter, Granulicatella, Grimontla, Haemophilus, Hahella, Halanaerobiumns, Haliangium, Halomonas, Halorhodospira, Halothermothrix, Halothiobacillus, Hamiltonella, Helicobacter, Heliobacterium, Herbaspirillum, Herminimonas, Herpetosiphon, Hippea, Hirschia, Histophilus, Hodgkinia, Hoelea, Holdemania, Hydrogenivirga, Hydrogenobaculum, Hylemonella, Hyphomicrobium, Hyphomonas, Idiomarina, Ilyobacter, Intrasporangium, Isoptericola, Isosphaera, Janibacter, Janthinobacterium, Jonesia, Jonquetella, Kangiella, Ketogulonicigenium, Kineococcus, Kingella, Klebslella, Kocuria, Koribacter, Kosmologa, Kribbella, Ktedonobacter, Kytococcus, Labrenzia, Lactobacius, Lactococcus, Laribacter, Lautropia, Lawsonia, Legionella, Leifsonia, Lentisphaera, Leptolyngbya, Leptospira, Leptothrix, Leptotrichia, Leuconostoc, Liberibacter, Limnobacter, Listeria, Loktanella, Lutiella, Lyngbya, Lysinibacillus, Macrococcus, Magnetococcus, Magnetospirillum, Mahella, Mannheimia, Maricaulis, Marinithermus, Marinobacter, Marinomonas, Mariprofundus, Maritimibacter, Marvinbryantla, Megasphaera, Meiothermus, Melissococcus, Mesorhizobium, Methylacidiphilum, Methylibium, Methylobacillus, Methylobacter, Methylobacterium, Methylococcus, Methylocystis, Methylomicroblum, Methylophaga, Methylophleales, Methylosinus, Methyloversatilis, Methylovorus, Microbacterium, Micrococcus, Microcoleus, Microcystis, Microlunatus, Micromonospora, Mitsuokella, Mobluncus, Moorella, Moraxella, Moritella, Mycobacterium, Myxococcus, Nakamurella, Natranaerobius, Neisserla, Neorickettsia, Neptuniibacter, Nitratifractor, Nitratiruptor, Nitrobacter, Nitrococcus, Nitrosomonas, Nitrosospira, Nitrospira, Nocardia, Nocardioides, Nocardiopsis, Nodularia, Nostoc, Novosphingobium, Oceanibulbus, Oceanicaulis, Oceanicola, Oceanithermus, Oceanobacillus, Ochrobactrum, Octadecabacter, Odyssella, Oligotropha, Olsenella, Opitutus, Oribacterium, Orientia, Omithinibacillus, Oscilatoria, Oscillochloris, Oxalobacter, Paenibacillus, Pantoea, Paracoccus, Parascardovia, Parasutterella, Parvibaculum, Parvimonas, Parvularcula, Pasteurellq, Pasteuria, Pectobacterium, Pediococcus, Pedosphaera, Pelagibaca, Pelagibacter, Pelobacter, Pelotomaculum, Peptoniphius, Peptostreptococcus, Persephonella, Petrotoga, Phaeobacter, Phascolarctobacterium, Phenylobacterium, Photobacterlum, Pirellula, Planctomyces, Planococcus, Plesiocystis, Polaromonas, Polaromonas, Polymorphum, Polynucleobacter, Poribacteria, Prochlorococcus, Propionibacterium, Proteus, Providencia, Pseudoalteromonas, Pseudoflavonifractor, Pseudomonas, Pseudonocardia, Pseudoramibacter, Pseudovibrio, Pseudoxanthomonas, Psychrobacter, Psychromonas, Puniceispkillum, Pusillimonas, Pyramidobacter, Rahnella, Ralstonia, Raphidiopsis, Regiella, Reinekea, Renibacterium, Rhizobium, Rhodobacter, Rhodococcus, Rhodoferax, Rhodomicrobium, Rhodopirellula, Rhodopseudomonas, Rhodospirillum, Rickettsia, Rickettsiella, Riesia, Roseburia, Roseibium, Roseiflexus, Roseobacter, Roseomonas, Roseovarius, Rothia, Rubrivivax, Rubrobacter, Ruegeria, Ruminococcus, Ruthia, Saccharomonospora, Saccharophagus, Saccharopolyspora, Sagittula, Salinispora, Salmonella, Sanguibacte, Scardovia, Sebaldella, Segniliparus, Selenomonas, Serratia, Shewanella, Shigella, Shuttleworthia, Sideroxydans, Silicibacter, Simonslella, Sinorhizoblum, Slackia, Sodalls, Solibacter, Solobacterium, Sorangium, Sphaerobacter, Sphingobium, Sphingomonas, Sphingopyxis, Spirochaeta, Sporosarcina, Stackebrandtla, Staphylococcus, Starkeya, Stenotrophomonas, Stigmatella, StreptobacWllus, Streptococcus, Streptomyces, Streptosporangium, Subdoligranulum, subvibrioldes, Succinatimonas, Sulptobacter, Sulfobacillus, Sulfuricurvum, Sulfurhydrogenibium, Sulfurimonas, Sulfurospirillum, Sulfurovum, Sutterella, Symbiobacterum, Synechocystis, Syntrophobacter, Syntrophobotulus, Syntrophomonas, Syntrophothermus, Syntrophus, talwanensis, Taylorella, Teredinibacter, Terriglobus, Thalassiobium, Thauera, Thermaerobacter, Thermanaerovibrlo, Thermincola, Thermoaneerobacter, Thermoanaerobacterum, Thermobaculum, Thermobifida, Thermobispora, Thermocrinis, Thermodesulphateator, Thermodesulfobacterlum, Thermodesulfoblum, Thermodesulfovibrlo, Thermomicrobium, Thermomonospora, Thermosediminibacter, Thermosinus, Thermosipho, Thermosynechococcus, Thermotoga, Thermovibrio, Thermus, Thloalkallmicrobium, Thioalkalivibrio, Thiobacillus, Thiomicrospira, Thiomonas, Tolumones, Treponema, tribocorum, Trichodesmium, Tropheryma, Truepera, Tsukamurella, Turicibacter, Variovorax, Veillonella, Verminephrobacter, Verrucomicroblum, Verrucosispora, Vesicomyosocius, Vibro, Vibrionales, Victivallis, Weissella, Wigglesworthia, Wolbachia, Wolinella, Xanthobacter, Xanthomonas, Xenorhabdus, Xylanimonas, Xylella, Yersinia, Zinderia and Zymomonas.

In particular, the recombinant cell may be selected from the group consisting of Bacillus subtilis, Burkholderia thailandensis, Corynebacterium glutamicum, Cyanobacteria, Escherichia coli, Klebsiella oxytoca, Pseudomonas fluorescens, Pseudomonas pulida, Pseudomonas stutzeri, and Rhizobium meliloti. These bacterial cells which do not naturally produce glycerol dehydratase and are readily available for genetic manipulations are suitable for the transformation with exogenous genes coding for a functional glycerol dehydratase. In a preferred aspect of the present invention, E. coli is used as a parent cell to construct a recombinant microbial biocatalyst for the production of 3-hyrdoxypropionaldehyde. In another preferred aspect of the present invention, the thermophilic bacterium Bacillus coagulans is used as a parent cell to construct a recombinant microbial biocatalyst for the production of 3-hyrdoxypropionaldehyde.

In another example, the recombinant cell may be selected from the group consisting of Citrobacter freundii, C. butyricum, C. acetobutylicum, E. agglomerans, L. reuteri, and K. pneumoniae. These bacterial cells naturally produce glycerol dehydratase and are further genetically modified to increase the glycerol dehydratase expression relative to the wild type cell. Seyfried M, et al. (1996) J. Bacteriol. 178, 5793-5796; Ulmer C, et al. (2007) Chem Biochem Eng Quart 21(4): 321-326, and van Pijkeren J-P, et al. (2012) Bioengineered 3:209-217 describe ways in which these bacterial cells which naturally produce glycerol dehydratase may be further genetically modified to increase the expression of glycerol dehydratase enzyme relative to the wild type cell.

The microbial biocatalysts used for production of 3-hydroxypropionaldehyde may be used as free or immobilized cells. In particular, the aqueous medium used according to any aspect of the present invention must be capable of maintaining the growth of these cells without being toxic to the cell. More in particular, the aqueous medium according to any aspect of the present invention accompanied by the fractional distillation process for acrolein recovery must be capable of maintaining the production of 3-hydroxypropionaldehyde without being toxic to the cell.

In the first step of construction of a recombinant microbial cell for producing 3-hydroxypropionate using glycerol as a feedstock, it is necessary to introduce a glycerol dehydratase gene if such a gene is not already present in the selected microorganism followed by the blocking of all other pathways for glycerol utilization.

There are two different types of glycerol dehydratase enzymes. The glycerol dehydratase enzyme present in L. reuteri has a subunit composition of α2β2γ2 and requires coenzyme B12 (cobalamin) for its activity. Analysis of complete sequence for L. reuteri and L. fermentum has identified the genes gupCDE coding for each of the subunits of B12-dependent glycerol dehydratase as well as the genes coding for the enzymes involved in cobalamin biosynthesis. The glycerol dehydratase present in Clostridium butyricum does not require coenzyme B12 for its activity and it is referred as B12-independent glycerol dehydratase. Both B12-dependent glycerol dehydratase and B12-independent glycerol dehydratase undergo suicidal inactivation and require an activating enzyme to reactivate the catalytic activity. The activating enzyme for B12-dependent glycerol dehydratase is a tetramer comprising two different subunits. The B12-independent glycerol dehydratase and its activating enzyme are encoded by the genes dhaB1 and dhaB2, respectively.

In one embodiment, the present invention provides a recombinant microorganism comprising exogenous genes coding for each of the three subunits of B12-dependent glycerol dehydratase as well as the genes coding for the enzymes involved in the biosynthesis of vitamin B12. The introduction of genes coding for enzymes involved in the biosynthesis of vitamin B12 will eliminate the requirement for supplementing the fermentation medium with expensive vitamin B12. In a preferred embodiment, the present invention provides a recombinant microorganism comprising exogenous dhaB1 and dhaB2 genes coding for B12-independent glycerol dehydratase and its activating enzyme.

In one aspect of the present invention, the exogenous genes coding for B12-dependent glycerol dehydratase or B12-independent glycerol dehydratase are introduced into an acidophilic microorganism which can be grown at an acidic pH so that the equilibrium between 3-hydroxypropionaldehyde and acrolein is tilted towards acrolein to facilitate the removal of acrolein through distillation process. The acid tolerant microbial organisms are typically isolated from acidic environment such as acidic bogs or corn steep water of a commercial corn milling facility. An acid tolerant microorganism which can also grow at elevated temperatures is preferred. A number of acidophilic yeast strains belonging to the genus Saccharomyces, Kluyveromyces and Issatchenkia have been developed for manufacturing a number of carboxylic acids such as lactic acid and succinic acid without the need for adding alkali material to maintain the pH of the culture medium during the production phase. Any one of those yeast strains can be used as a host microbial cell to express one or other exogenous glycerol dehydratase genes for the purpose of producing 3-hydroxypropionaldehyde. Similarly, a number of strains of Lactobacillus reuteri have been reported to be tolerant to acid conditions as low as pH 3.0. A number of Escherichia coli bacterial strains genetically engineered to produce one or other organic acids are also known to have tolerance to low pH growth conditions. Any one of those acid-resistant bacterial strains can be used as a host cell in developing acidophilic recombinant host cells of the present invention. In the preferred embodiment, the acidophilic microorganisms harboring the exogenous glycerol dehydratase enzyme may further comprise mutations that block activity of the enzymes that functions in the other pathways for glycerol utilization such as propionic acid pathway, dihydroxyacetone pathway and 1, 3-propanediol pathway (FIG. 1).

In another aspect of the present invention, the glycerol uptake by the microorganism selected for the production of 3-hydroxypropionaldehyde and recovery of acrolein according to the present invention is further improved. In general, glycerol uptake from the culture medium by a microorganism occurs through a passive diffusion process. In some organisms, the glycerol uptake by the microorganism is facilitated by one or more proteins located in the outer membrane. In one aspect of the present invention, where the microorganism, selected for production of 3-hydroxypropionaldehyde using glycerol as a feedstock, contains a gene coding for a protein facilitating glycerol uptake, the expression of that gene can be further increased through appropriate genetic manipulations to further improve the glycerol uptake. For example, in certain Lactobacillus strains, the pduP gene codes for a protein facilitating the uptake of glycerol. By means of expressing the pduP gene under a stronger promoter, the glycerol uptake by the microorganism can be improved. When the microorganism selected for the production of 3-hydroxypropionaldehyde from glycerol, does not have any endogenous genes coding for the protein facilitating the glycerol uptake, an exogenous gene coding for protein that facilitate the glycerol uptake such as pduP or glpF gene can be introduced to improve the glycerol uptake in the selected microorganism.

In one aspect of the present invention, an exogenous B12-dependent glycerol dehydratase enzyme is introduced into the acidophilic microorganism. In another aspect of the present invention, an exogenous B12-independent glycerol dehydratase enzyme is introduced into the acidophilic microorganism. In a preferred aspect of the present invention, besides introducing an exogenous B12-independent glycerol dehydratase enzyme into the acidophilic microorganism, various glycerol utilization pathways other than the 3-hydroxypropanaldehyde pathway that exist within the acidophilic microorganism are blocked through appropriate genetic modifications.

In yet another preferred embodiment of the present invention, the exogenous genes coding for B12-dependent glycerol dehydratase or B12-independent glycerol dehydratase are introduced into a thermophilic microorganism which can be grown at an elevated temperature. In the present invention, distillation process is followed to recover acrolein from the fermentation broth. Acrolein has a boiling point of 53° C. and in order to reduce the boiling point the vapor pressure within the fermentation vessel is lowered so that the distillation can be carried out at a temperature much lower than 53° C. However, by means of growing recombinant microorganism at an elevated temperature, the distillation process can be carried out at elevated temperature without the need to reduce the vapor pressure within the fermentation vessel significantly. A number of microbial cells including Bacillus coagulans and Caloromator viterbenis are known to grow at elevated temperature. Any one of those thermophilic microorganisms can be used as a host microbial cell to express one or other exogenous glycerol dehydratase genes for the purpose of producing 3-hydroxypropionaldehyde. In the preferred embodiment, the thermophilic microorganisms harboring the exogenous glycerol dehydratase enzyme may further comprise mutations that block activity of the enzymes that functions in the other pathways for glycerol utilization such as propionic acid pathway, dihydroxyacetone pathway and 1, 3-propanediol pathway (FIG. 1).

To facilitate a better understanding of the present invention, the following examples of preferred or representative embodiments are given. In no way should the following examples be read to limit, or to define, the scope of the invention.

### EXPERIMENTAL SECTION

### Analytical Techniques:

Acrolein test was used for quantitative analysis of 3-hydroxypropionladehyde. 200 μl of a suitably diluted sample was mixed with 600 μl HCl for the dehydration of 3-HPA to acrolein. DL-tryptophan (150 μl) was added to the mixture, thereby obtaining an acrolein-chromophore complex (purple) which was quantified by absorbance at 560 nm on a spectrophotometer using acrolein as standard (Vollenweider, S., et al., Journal of Agricultural and Food Chemistry, 2003, 51(11): pp. 3287-3293; Circle, S. Ind Eng Chem Anal Ed, 1945. 17: pp. 259-262).

### Example 1

### Determining the Optimum pH and Temperature for Acrolein Formation During Fermentation and for Acrolein Recovery

The present invention provides a method for fermentation process involving microbial catalyst with the capacity to produce 3-hydroxypropionaldehyde from glycerol feedstock. The 3-hydroxypropionaldehyde resulting from the fermentation process accumulates in the fermentation broth and undergoes spontaneous dehydration reaction to yield acrolein. In the course of fermentation, 3-hydroxypropionaldehyde and acrolein are expected to reach a chemical equilibrium and the relative molar concentration of 3-hydroxypropionaldehyde and acrolein is expected to vary depending on the temperature and pH of the fermentation broth. An accumulation of 3-hydroxypropionaldehyde in the fermentation broth beyond a certain limit is toxic to the microbial cells and there is a need in the field to remove the 3-hydroxypropionaldehyde as soon as it is formed to maintain the continuous fermentation process. The present invention provides an in-situ continuous process to remove acrolein from the fermentation broth using fractional distillation. Such a continuous removal of acrolein through fractional distillation is expected to maintain the concentration of 3-hydroxypropionaldehyde at a level not toxic to the microbial cells.

The spontaneous conversion of 3-hydroxypropionaldehyde to acrolein has been reported to be enhanced by an elevated temperature and a lowered pH. However, the optimal pH and temperature range for the spontaneous conversion of 3-hydroxypropionaldehyde to acrolein is not known. The objective of these experiments is to determine the optimal pH and temperature range for fermentation process and the acrolein recovery through fractional distillation. More specifically, these experiments aim to determine the relative molar concentrations of acrolein and 3-hydroxypropionaldehyde under different chemical equilibria at different pH and temperature.

3-hydroxypropionaldehyde is chemically synthesized by mixing 7.5 mL acrolein (92% v/v) with 32.5 mL H2O and 10 mL H2SO4 (1.5 M) and incubating the mixture in the dark for two hours at 50° C. After cooling down to 4° C. the pH was adjusted to 6.8 by adding 5 M NaOH and undesired by-products (derivates) and remaining acrolein were extracted with chloroform (Vollenweider, S., Grassi, G., K'onig, I., Puhan, Z., Purification and structural characterization of 3-hydroxypropionaldehydend its derivatives. J. Agric. Food Chem. 2003, 51: 3287-3293). 3-hydroxypropionaldehyde concentration is determined by using the Circle's method (Acrolein Determination by Means of Tryptophane—A Colorimetric Micromethod. CIRCLE, S. D., STONE, L., and BORUFF, C. S. 1945, Industrial and Engineering Chemistry, 17: 259-262).

Quantification of 3-hydroxypropionaldehyde is done indirectly by converting it into acrolein through acid treatment and quantifying the acrolein using a colorimetric assay. Samples are centrifuged in a centrifuge at 13,300 rpm and 4° C. for 5 min to remove solids. 250 μL sample from supernatant cooled in iced water is mixed with 500 μL HCl 37% at 4° C. and 125 μL DL-tryptophan solution at 4° C. Optical density is measured at 560 nm immediately after incubation at 37° C. for 40 min. The use of HCl shifts the reaction equilibrium between 3-hydroxypropionalde and acrolein completely to acrolein. Standard curves are obtained with 0-10 mM acrolein in aqueous solution using the same procedure.

In the first experiment, 1 ml of 1 molar solution 3-hydroxypropionaldehyde is aliquoted into a test tube with 4 ml of an aqueous solution with different pH (3.5, 4.0, 4.5, 5.0, 5.5, 6.0, 6.5, 7.0, 7.5 and 8.0). These test tubes are incubated at room temperature for one hour and tested for the molar concentration of 3-hydroxypropionaldehyde and acrolein concentration. In determining the 3-hydroxypropionaldehyde, 250 μL sample from a test tube cooled in iced water is mixed with 500 μL HCl 37% at 4° C. and 125 μL DL-tryptophan solution at 4° C. Acrolein concentration is determined using a GC/MS method (see Worldwide Website: epa.gov/sites/production/files/2015-08/documents/method_603_1984.pdf).

Once an appropriate pH is determined where 3-hydroxypropionaldehyde and acrolein are in equimolar concentration, that particular pH is used for testing the effect of fractional distillation on the relative molar concentration of 3-hydroxypropionaldehyde and acrolein. In this experiment, 3-hydroxypropionaldehyde is taken up in large volume and subjected to fractional distillation at 53° C. At periodic intervals, samples are collected from the reaction vessel and the relative molar concentration of 3-hydroxypropionaldehyde and acrolein are determined. In another set of experiment, fractional distillation is carried out at 35° C. while reducing the vapor pressure within the reaction vessel to varying levels. At periodic intervals, samples are removed from the reaction vessels with different internal vapor pressure and the relative molar concentration of 3-hydroxypropionaldehyde and acrolein are determined. The results from these two sets of experiments are analyzed to decide on the optimal pH, temperature and vapor pressure for carrying out the glycerol fermentation process as well as the fractional distillation process for recovering acrolein from fermentation broth.

### Example 2

### Lactobacillus reuteri DSM 20016 Strain for Bio-3-Hydroxypropionaldehyde Production

L. reuteri DSM 20016 strain is selected for this initial study to determine the level of 3-hydroxypropionaldehyde induced toxicity and the suitability of the fractional distillation for recovering acrolein according to the present invention. L. reuteri DSM 20016 strain is reported to have two different open reading frames namely lr-0030 and lr-1734 coding of 1,3-propanediol dehydrogenase. Mutagenic analysis has indicated that the open reading frame lr-0030 codes for 1,3-propanediol dehydrogenase that is active during the exponential growth phase and the open reading frame lr-1734 codes for 1,3-propanediol dehydrogenase that is active during the 3-hydroxypropionaldehyde production phase. Wild type L. reuteri DSM 20016 strain as well as the two mutant strains namely L. reuteri DSM 20016—Δlr-0030 and L. reuteri DSM 20016—Δlr-1734 are tested in the present investigation. All three strains are grown in MRS medium containing 35 mM glycerol. When the cell density reaches an OD₆₀₀ of 8, the cells are collected, washed and resuspended in aqueous medium containing 250 mM glycerol at an OD₆₀₀ of 60 at 37 C. The microbial cells in the aqueous medium containing glycerol is subjected to distillation under vapor pressure of 62 mbar for one hour. Partial condensation is set at 25° C. and the distillate was collected in the trap cooled with liquid nitrogen to prevent uncondensed acrolein getting sucked into vacuum pump. The concentration of acrolein in the trap is determined using the standard method for assaying acrolein using DL-tryptophan as a coloring reagent. 250 μl of the sample was mixed with 500 μl of HCL 37% and 125 μl of DL-tryptophan and incubated at 37° C. for 40 minutes and the optical density was measured at 560 nm using a spectrophotometer. The standard curve was generated using acrolein in the concentration range of 0-10 mM.

### Example 3

### Escherichia coli Strain Expressing B-12 Dependent Glycerol Dehydratase

When a microbial cell lacking endogenous glycerol dehydratase enzyme is selected for development as a microbial catalyst for the production of bioacrolein according to the present invention, it is necessary to introduce an exogenous glycerol dehydratase enzyme into the selected microbial strain. Ilyobacter polytropus, Klebsiella pneumoniae, Citrobacter freundii and the like, are used as a source for a B12-dependendent glycerol dehydratase enzyme. The Ilyobacter polytropus-derived glycerol dehydratase has 3 structural subunits namely DhaB1, DhaB2 and DhaB3 making up the a, p and 7 subunits of a B12-dependent glycerol dehydratase enzyme. Glycerol dehydratase from Klebsiella pneumoniae and Citrobacter freundii contains 3 structural subunits namely DhaB, DhaC and DhaE (SEQ ID NO: 1, SEQ ID NO: 2 and SEQ ID NO: 3). These three subunits make up the a, 0 and 7 subunits of a B12-dependent glycerol dehydratase enzyme.

The B12-dependent glycerol dehydratase is irreversibly inactivated by glycerol and the recombinant microorganism receiving an exogenous B12-dependent glycerol dehydratase needs to have an exogenous gene coding for glycerol dehydratase reactivator for activating glycerol dehydratase as well. The nucleic acid sequence encoding a dehydratase reactivator is selected from Citrobacter freundii (dhaFG), Klebsiella pneumonia (gdrAB), Klebsiella oxytoca (ddrAB), Ilyobacter polytropus (gdrA and gdrB) and the like (SEQ ID NO: 4 and SEQ ID NO: 5 provide exemplary amino acid sequences).

### Example 4

### Escherichia coli Strain Expressing B-12 Independent Glycerol Dehydratase

The bacterial strain Clostridium butyricum VPI 1718 is used as a source for the genes coding for B12-independent glycerol dehydratase and its reactivator (dhaB1B2, SEQ ID NO: 6 and SEQ ID NO: 7). Escherichia coli DH5a (E. coli DH5a) and E. coli BL21 (DE3) are used as host strains for cloning and expression of the genes dhaB1B2, respectively. Plasmid pMD18-T vector is used for cloning the dhaB1B2 genes, and plasmid pET-22b (+) is used as a vector for expressing the dhaB1B2 genes cloned from C. butyricum.

The dhaB1B2 genes are cloned based on the polymerase reaction (PCR) with the forward primer K1 (5-GCGCCATIGGTAAGTAAAGGATTTAGTACCC-3, SEQ ID NO: 8) with NcoI restriction site and the reverse primer K2 (5-CGGGATCCTATTACTCAGCTCCAATTGT-3, SEQ ID NO: 9) with BamHI restriction site. After the PCR, all products are identified by 1% agarose gel electrophoresis. PCR products are purified by gel purification before reassembling into plasmid vectors pMD18-T and pET-22b (+).

E. coli strain DE3 is transformed with pET-22-dhaB1B2 plasmid and transformed cells are assayed for glycerol dehydratase expression. DE3 strain harboring the recombinant plasmid is inoculated into LB liquid medium with ampicillin (75 μg/mL) and incubated at 37° C. overnight. Subsequently, the mixture is transferred to fresh LB liquid medium (1:100 dilution) containing ampicillin (100 μg/mL) and cultured at 37° C. for 2 h. At an optical density (OD600) of 0.5-0.6, IPTG is added to the final concentration of 1 mM, and the mixture is incubated at 37° C. for 5 h. Then, the cells are harvested and the lysozyme is added (final concentration 10 mg/mL), incubated at 37° C. for 1 min, and finally centrifuged for 10 min at 10,000 g/min to collect supernatant. The glycerol dehydratase activity is measured with 1, 2-propanediol as a substrate previously described by Daniel et al., with a moderate modification. The MBTH method is used for measuring glycerol dehydratase activity: the principle is based on the fact that glycerol dehydratase can catalyze the conversion of 1, 2-propanediol to propionaldehyde, and the reaction of propionaldehyde with MBTH can generate triazine that can be detected by a spectrophotometer at 305 nm.

### Example 5

### Acidophilic Yeast Strain Expressing B-12 Independent Glycerol Dehydratase

Low pH tolerant yeast strains have been developed for producing carboxylic acid at industrial scale. Saccharomyces cerevisiae, Kluyveromyces marxianus, Issatchenkia orientalis, and Yarrowia lipolytica strains have been genetically engineered to produce succinic acid at relatively low pH (WO 2008/128522; WO 2010/043197; US 2012/0040422; WO 2010/003728; WO 2011/023700; WO 2009/101180; WO 2012/038390; WO 2012/103261; WO 204/043591 and US 2012/0015415). Any one of these low pH tolerant yeast strains is suitable for 3-hydroxypropionaldehye production using glycerol as a feedstock.

In the first step in developing a yeast strain for 3-hydroxypropionaldehye production using glycerol as a feedstock, an exogenous gene coding for glycerol dehydratase enzyme is introduced into the selected yeast cell using readily available genetic engineering techniques. Depending on the presence or absence of the genes for vitamin B12 biosynthesis in the selected low pH tolerant yeast strain, one can choose to introduce exogenous genes coding for B12-dependent glycerol dehydratase enzyme or B12-independent glycerol dehydratase enzyme. If the low pH tolerant yeast strain selected for 3-hydroxypropionaldehye production using glycerol as a feedstock already possess an endogenous glycerol dehydratase enzyme, one should consider enhancing the activity of the endogenous glycerol dehydratase by means of increasing the expression of endogenous glycerol dehydratase.

After assuring that the there is a fully-functional glycerol dehydratase with in the selected low-pH tolerant yeast strain, efforts are made to block the competing glycerol utilization pathway in the yeast cell by means of inactivating the genes coding for NADH-dependent oxidoreductase, NAD-linked glycerol dehydrogenase, dihydroxyacetone kinase and aldehyde dehydrogenase. Once required genetic modifications are accomplished in the yeast strain with low-pH tolerance, it is used in the two-step fermentation process to produce 3-hydroxypropionaldehyde using glycerol as a feedstock. In the first-stage, the selected yeast strain is grown in a glucose containing medium to go through an exponential growth phase to accumulate required cell mass. In the second stage, the cells from the exponential growth phase are harvested, washed and resuspended at a very high cell density in a slightly acidic aqueous solution containing glycerol to initiate the 3-hydroxypropionaldehyde production. During the 3-hydroxypropionaldehyde production phase, the fermentation vessel is maintained at a reduced vapor pressure to facilitated the distillation of the acrolein resulting from the spontaneous dehydration of 3-hydroxypropionaldehyde at a slightly acidic pH prevailing in the fermentation vessel. The acrolein removed from the fermentation vessel through distillation is collected in a trap maintained at a low temperature.

### Example 6

### Bacillus coagulans Expressing B-12 Independent Glycerol Dehydratase

Bacillus coagulans strain P4-102B grows optimally at 50° C. and pH5. L-broth (LB) is used as the rich medium to culture this bacterium at pH 5.0 or 7.0, as needed. Glucose is sterilized separately and added to the medium before inoculation. Chloramphenicol, erythromycin, and ampicillin are added to LB medium at 7.5 mg L-1, 5 mg L-1, and 100 mg L-1, respectively, when needed.

Plasmid pGK12 carries chloramphenicol and erythromycin-resistance genes and is useful in transforming B. coagulans. Plasmid pGK12 and its derivatives are maintained in B. subtilis strain HB1000 at 37° C. When transformed into B. coagulans, the transformants were selected and maintained at 37° C. The replication of the plasmid pGK12 is naturally restricted to temperatures ≤42° C. This temperature sensitive nature of plasmid pGK12 replication at 50° C. provides an opportunity to select for chromosomal DNA integrants of B. coagulans that can grow at 50-55° C.

The following procedure is used for transformation of wild type B. coagulans P4-102B. Cells growing in 10 mL of LB in a 125 mL flask at 50° C. (OD420 nm 0.3) is inoculated (10% vol/vol) into 100 mL of LB medium in a 1 liter flask. Cells are incubated at 50° C. with shaking (200 rpm) for about 3-4 h until the OD at 420 nm reached about 0.3-0.5. Cells are collected by centrifugation (4° C.; 4;300×g; 10 min) and washed three times with 30, 25, and 15 mL of ice-cold SG medium (sucrose, 0.5 M, glycerol, 10%). These electro-competent cells are used immediately. The cell suspension (75 μL) is mixed with 0.1 μg of plasmid DNA and transferred to chilled electroporation cuvette (1 mm gap). The electroporation condition is set as square wave for 5 ms at 1.75 KV (BioRad electroporator; BioRad Laboratories, Hercules, CA). After electroporation, cells are transferred to 2 mL of prewarmed (37° C. or 50° C.) RG medium (LB medium with 0.5 M sucrose, 55.6 mM glucose and 20 mM MgCl2). These cells are transferred to a 13×100 mm screw cap tube and incubated in a tube rotator for 3 h at 50° C. before plating on selective antibiotic medium.

Using the plasmids and transformation procedure described in this Example, genes coding for B12-independent glycerol dehydratase and its reactivator derived from Clostridium butyricum are introduced into the Bacillus coagulans strain P4-102B to facilitate the production of 3-hydroxypropionaldehyde using glycerol as a feedstock.

Using the same plasmid system and the transformation protocol, it is possible to delete any genes involved in any other endogenous glycerol utilization pathway in this bacterial strain for the purpose of improving the production of 3-hydroxypropionaldehyde from glycerol. Since this bacterial strain is able to grow optimally at 50° C. and pH5, the spontaneous conversion of 3-hydroxypropionaldehyde to acrolein occurs at much greater efficiency when compared to the E. coli stain harboring the B12-independent glycerol dehydratase and its reactivator derived from Clostridium butyricum. Increased efficiency for the spontaneous conversion of 3-hydroxypropionaldehyde to acrolein is the most desirable feature for the production and recovery of bioacrolein according to the present invention.

### Example 7

### Calormator Viterbenis Cells as a Source of Thermophilic Glycerol Dehydratase

Examples of thermophilic microorganisms include members of the genera Bacillus, Thermus, Sulfolobus, Thermoanaerobacter, Thermobrachium, and Caloramator. The Caloramator viterbenis JW/MS-VS5^{T} (ATCC PTA-584) strain was isolated from a mixed sediment/water sample collected from a freshwater hot spring in the Bagnaccio Spring area near Viterbo, Italy, in June 1997. The cells of this strain occur singly and stain Gram positive. The temperature range for growth at pH 6.0 is 33-64° C., the optimum at 58° C. The pH range for growth is from 5.0 to 7.6, with an optimum at 6.0-6.5.

Using the nucleotide probes based upon glycerol dehydratase gene from a non-thermophilic organism (e.g., K. pasfeurianum, C. freundii, or C. pasteuriamum), such as the dhaBCE genes, which encode glycerol dehydratase corresponding homologous gene sequences is obtained for Caloramator viterbenis and used as the source of thermophilic glycerol dehydratase in the thermophilic microbial strains such as Bacillus coagulans.

### Example 8

### Method for Bioacrolein Biosynthesis

A two-step process is used for high level of 3-hydroxypropionaldehyde production and its subsequent conversion to acrolein through spontaneous dehydration reaction. Any one of the microbial strains described in the Examples 2-7 above with the appropriate genetic modifications in the glycerol utilization pathway is used in this two-step fermentation process. The appropriate genetic modification in the glycerol utilization pathway encompasses an increase in the activity of glycerol dehydratase enzyme and inhibition of NAD-linked glycerol dehydrogenase, NADH-dependent oxidoreductase and aldehyde dehydrogenase.

Cells of the selected microbial strain are first propagated overnight in optimal conditions for cell growth in a minimal medium with glucose as a source of carbon. Difco™ Lactobacilli MRS Broth powder containing peptone and dextrose is used to grow Lactobacillus reuteri stains. The ingredients in MRS broth supply nitrogen, carbon and other elements necessary for growth. Polysorbate 80, acetate, magnesium and manganese in MRS broth provide growth factors for culturing a variety of lactobacilli. The above ingredients may inhibit the growth of some organisms other than Lactobacilli. The cells grown in glucose containing medium are harvested, washed and incubated in a pure aqueous glycerol solution to initiate the 3-hydroxypropionaldehyde.

3-hydroxypropionaldehyde production in the glycerol containing medium is optimized with reference to biomass concentration, temperature, oxygen level, glycerol concentration and incubation time. Cell viability and 3-hydroxypropionaldehyde concentration are measured over time during glycerol bioconversion to 3-hydroxypropionaldehyde to study the toxicity of 3-hydroxypropionaldehyde towards the production strain itself. The feasibility of reusing 3-hydroxypropionaldehyde producing cells is investigated by successive cell transfer to fresh glycerol containing medium.

### Example 9

### Recovering Bioacrolein Using Distillation Process

Any one of the microbial catalysts described in the Examples 1-8 is suitable for 3-hydroxypropionaldehyde production using glycerol in commercial scale. The preferred fermentation protocol involves two-step process. In the first step of the fermentation process, the selected microbial biocatalysts in a fermentation broth containing readily metabolizable carbon source such as glucose undergoes an exponential growth phase. At the end of the exponential growth phase, the cell mass is collected, washed and resuspended at higher cell density in an aqueous medium containing glycerol to initiate the production phase. During the production phase, also referred as the second stage of the fermentation process, glycerol is converted into 3-hydroxypropionaldehyde. Depending on the pH and temperature within the fermentation vessel,3-hydroxypropionaldehyde undergoes spontaneous dehydration reaction leading to the production of acrolein. Since 3-hydroxypropionaldehyde and acrolein are in a chemical equilibrium, removal of the acrolein will allow the continuous production of 3-hydroxypropionaldehyde and assure neither 3-hydroxypropionaldehyde nor acrolein accumulate to a cytotoxic level within the biocatalysts. Thus, the present invention provides a continuous fermentation process for producing 3-hydroxypropionaldehyde based on an in-situ process for recovering acrolein using a fractional distillation process.

The removal of acrolein from the fermentation vessel is accomplished using a distillation which makes use of the much lower boiling point for acrolein when compared to the boiling point of 3-hydroxypropionaldehyde. Since the boiling point of a chemical entity depends on the vapor pressure, by means of reducing the vapor pressure within the fermentation vessel to a lower than that of atmospheric pressure (1,013.25 millibars) it is possible to lower the boiling point of all the chemical entities present within in the fermentation broth.

The microbial cells in the aqueous medium containing glycerol is subjected to distillation under vapor pressure of 62 mbar for one hour. Partial condensation was set at 25° C. and the distillate is collected in the trap cooled with liquid nitrogen to prevent uncondensed acrolein getting sucked into vacuum pump. The concentration of acrolein in the trap is determined using the standard method for assaying acrolein using DL-tryptophan as a coloring reagent. 250 μl of the sample was mixed with 500 μl of HCL 37% and 125 μl of DL-tryptophan and incubated at 37° C. for 40 minutes and the optical density was measured at 560 nm using a spectrophotometer. The standard curve was generated using acrolein in the concentration range of 0-10 mM. By means of determining the initial and final concentrations of glycerol, 3-hydroxypropionaldehyde and acrolein, the yield and specificity for both 3-hydroxypripionaldhyde and acrolein are determined.

### Example 10

### Manufacturing Bioacrylic Acid Using Bioacrolein as a Feedstock

Bioacrolein recovered from the fermentation broth using fractional distillation process according to the present invention is subjected to oxidation involving heterogeneous catalysts to produce bioacrylic acid. The heterogeneous catalysts useful in the manufacturing acrylic acid using acrolein as a feedstock are known as multimetal oxides and comprise the elements of Mo and V. These multimetal oxide catalysts useful for the oxidation of acrolein to acrylic acid in commercial scale have been described in detail in the U.S. Pat. Nos. 3,775,474; 3,954,855; 3,893,951; 4,339,355; and 7,211,692. Anyone of the multimetal oxide catalyst proven to be efficient and cost-effective in the acrylic acid industry is useful in the oxidation bioacrolein of the present invention to acrylic acid. One of the key criteria for selecting a multimetal oxide catalyst for the conversion bioacrolein to bioacrylic acid is its specificity for bioacrylic production.

In the traditional acrylic acid manufacturing from petrochemical feedstocks, two-step process is followed. In the first step of the acrylic acid manufacturing process, acrolein is derived from the propylene oxidation process along with a number of impurities such as furfural, maleic anhydride, maleic acid, formaldehyde and benzaldehyde. On the other hand, in the instant invention, the acrolein feedstock is derived from an aqueous solution containing only glycerol as the feedstock in a simplified fermentation process using a microbial organism. The conversion of glycerol to 3-hydroxypropionaldehyde is carried out by a single enzymatic reaction. 3-hydroxypropionaldehyde is the only product resulting from the glycerol fermentation according to the present invention and it undergoes spontaneous dehydration reaction to acrolein which is recovered using fractional distillation process. During the conversion of glycerol to bio-3-hydroxypropionaldehyde within the microbial biocatalyst, no other major metabolic pathways are functional. Consequently, there is no accumulation of any major bye-products which would make it difficult to recover bioacrolein free of any impurities. Moreover, bioacrolein with a boiling point of 53° C. is recovered from the fermentation as soon as it is formed by fractional distillation. Further by means of reducing the pressure less than that of atmospheric pressure (1,013.25 millibars) within the fermentation vessel, the temperature for the fractional distillation of the acrolein may further be reduced as low as 37° C. Alternatively, by means of using acidophilic microbial biocatalysts for the production of 3-hydroxypropionaldehyde, the high temperature requirement for the fractional distillation of acrolein may further be lowered. Since there is neither a high-temperature inactivation nor any acid precipitation step in the bioacrolein recovery, there no protein or nucleic acid degradation within the biocatalyst. As a result, the impurities such as nitrogen, and sulfur generally associated with the organic products derived from biological fermentation using high-temperature treatment and acid precipitation steps are absent in the bioacrolein manufactured according to the present invention.

In the traditional acrylic acid manufacturing plant using petrochemical feedstock, acrolein produced in the first reactor has a tendency to form explosive mixture with air and stem is used as a diluent in the second reactor where acrolein is oxidized to acrylic acid. During the fractional distillation process according to the present invention, acrolein is expected to azeotrope with water (2.5-3.0% with acrolein azeotrope boiling point of 52.4° C.)), see homepages.ed.ac.uk/jwp/Chemeng/azeotrope/AA.html. As a result, there is water in the overhead of the acrolein recovering unit to prevent the formation of an explosive mixture of air and acrolein besides making it easier to feed the acrolein stream of the present invention directly into the acrolein oxidizing unit of the existing acrylic acid plant.

Presence of water in the bioacrolein obtained using fractional distillation process may create some equilibrium back to 3-hydroxypropionaldehyde in the distillate but when it is fed into the second acrylic acid oxidation reactor, 3-hydroxypropionaldehyde will also oxidize to acrylic acid. In addition, the presence of water in the bioacrolein stream will have a beneficial use in the further processing of the bioacrolein to acrylic acid via oxidation. Due to the highly exothermic oxidation reaction, the current industrial process requires diluting the acrolein feed into the second oxidation reactor with steam. In the process according to the present invention, bioacrolein already comes with 2.5% water in it and therefore the presence of this water is not a detriment to the oxidation of bioacrolein.

### REFERENCES

All references are listed for the convenience of the reader. Each reference is incorporated by reference in its entirety.
U.S. Pat. No. 3,775,474
U.S. Pat. No. 3,893,951
U.S. Pat. No. 3,954,855
U.S. Pat. No. 4,339,355
U.S. Pat. No. 4,962,027
U.S. Pat. No. 5,821,092
U.S. Pat. No. 6,803,218
U.S. Pat. No. 7,211,692
U.S. Pat. No. 7,267,972
U.S. Pat. No. 7,410,754
U.S. Pat. No. 7,629,121
U.S. Pat. No. 7,695,943
U.S. Pat. No. 7,858,355
U.S. Pat. No. 7,985,849
U.S. Pat. No. 8,338,148
U.S. Pat. No. 8,445,659
U.S. Pat. No. 8,569,469
U.S. Pat. No. 8,609,904
U.S. Pat. No. 8,900,835
U.S. Pat. No. 8,951,763
U.S. Pat. No. 9,090,919
U.S. Pat. No. 9,365,875
U.S. Pat. No. 9,422,379
U.S. Pat. No. 9,587,255
U.S. Pat. No. 9,745,556
U.S. Pat. No. 10,041,094
U.S. Pat. No. 10,047,381
U.S. Pat. No. 10,053,411
U.S. Patent Application Publication No. 2005/004238
U.S. Patent Application Publication No. 2008/0131945
U.S. Patent Application Publication No. 2019/0365640
International Patent Application Publication No. WO 2012/033845
International Patent Application Publication No. WO 2013/134385
International Patent Application Publication No. WO 2014/043591
International Patent Application Publication No. WO 2015/034948
International Patent Application Publication No. WO 2016/201181
Cleusix, V., Lacroix, C., Vollenweider, S. and Duboux, M. (2007) Inhibitory activity spectrum of reuterin produced by Lactobacillus reuteri against intestinal bacteria BMC Microbiology 7: Article 101.
Daniel, R., Thomas A. Bobik, T. A. and Gerhard Gottschalk, G. (1999) Biochemistry of coenzyme B12-dependent glycerol and diol dehydratases and organization of the encoding genes. FEMS Microbio Rev. 22:553-566.
Doleyres, Y., Beck P., Vollenweider, S. and Lacroix, C. (2005) Production of 3-hydroxypropionaldehyde using a two-step process with Lactobacillus reuteri. Appl Microbiol Biotechnol (2005) 68:467-474.
Engels, C Schwab, C., Zhang, J., Stevens, M. J. A., Bieril, C., Ebert, M-O., McNeill, K., Sturla, S. J. and Lacroix, C. (2016) Acrolein contributes strongly to antimicrobial and heterocyclic amine transformation activities of reuterin. Sci. Rep. 6: 36246. doi.org/10.1038/srep36246.
Jers, C., Kalantari, A., Garg, A., and Mijakovic, I. (2019) Production of 3-hydroxypropanoic acid from glycerol by metabolically engineered bacteria. Frontiers in Bioengineering and Biotechnology, Volume 7: Article 124.
Jolly, J., Hitzmann, B., Ramalingam, S. and Kadathur B. Ramachandran, K. B. (2014) Biosynthesis of 1,3-propanediol from glycerol with Lactobacillus reuteri: Effect of operating variables. J. BioSci. Bioeng. 118:188-194.
Kandasamy, V., Hema Vaidyanathan, H., Djurdjevic, I., EJayamani, E., Ramachandran, K. B., Wolfgang Buckel, W., Jayaraman, G. and & Ramalingam, S. (2013) Engineering Escherichia coli with acrylate pathway genes or propionic acid synthesis and its impact on mixed-acid fermentation. Appl. Microbiol. Biotechnol. 97:1191-1200.
Knietsch, A., Bowien, S., Whited, G., Gerhard Gottschalk, G. and Rolf Daniell, R. (2003) Identification and Characterization of Coenzyme B12-Dependent Glycerol Dehydratase- and Diol Dehydratase-Encoding Genes from Metagenomic DNA Libraries Derived from Enrichment Cultures. App. Env. Microbiol. 69:3048-3060.
Kovaçević, B., Baric, D., Babić, D., Bilić, L., Hanževački, M., Gregory M. Sandala, G., Radom, L. and Smith, D. M. (2018) A Computational Tale of Two Enzymes: Glycerol Dehydration With or Without B12. J. Amer. Chem. Soc., 140:8487-8496.

Kristjansdottir, T., Elleke F. Bosma, E. F., Branco dos Santos, F., Özdemir, E., Herrgard, M. J., França, L., Ferreira, B., Nielsen, A. T. and Gudmundsson, S. (2019) A metabolic reconstruction of Lactobacillus reuteri JCM 1112 and analysis of its potential as a cell factory. Cell Fact, 18:186-205.
Morital, H., Toh, H., Fukuda, S., Horikawal, H., Oshima, K., Suzuki, T., MurakamI, M., Hisamatsu, S., kato, Y., Takizawa, T., Fukuoka, H., Yoshimura, T., Itoh, K., O'Sullivan, D. J. O., McKay, L. L., Ohno, H., Kikuchi, J., Masaoka, T., and Hattori, M. (2008) Comparative genome analysis of Lactobacillus reuteri and Lactobacillus fermentum reveal a renomic island for reuterin and cobalamin production. DNA Res. 15. 151-161.
Motaa, M. J., Lopesa, R. P., Sousab, S., Gomesb, A. M. Delgadilloa, I. and Saraivaa, J A. (2018) Lactobacillus reuteri growth and fermentation under high pressure towards the production of 1,3-propanediol. Food Res. Int. 113: 424-432.
Nakamura, C. E. and Whitedy, G. M. (2003) Metabolic engineering for the microbial production of 1,3-propanediol. Curr. Opin. Biotech. 14:454-459.
Oehmke, S. and Zeng, A-P. (2015) Recovery of biologically produced 3hydroxypropionaldehyde and its dehydrated product acrolein. Eng. Life Sci. 15:133-139.
Raynaud, C., Sarcabal, P., Meynial-Salles, I., Croux, C. and Soucaille, P. (2003) Molecular characterization of the 1, 3-propanediol (1, 3-PD) operon of Clostridium butyricum. Proc. Natl. Aca. Sci. 100:5010-5015.
Rodriguez, E., Arque's, J. L., Rodriguez, R., Nunez, M, and Medina, M. (2003) Reuterin production by lactobacilli isolated from pig faeces and evaluation of probiotic traits Lett. App. Microbio. 37: 259-263.
Stevens, M. J. A., Vollenweider, S., Meile, L. and Lacroix, C. (2011) 1, 3-Propanediol dehydrogenases in Lactobacillus reuteri: impact on central metabolism and 3-hydroxypropionaldehyde production. Microbial Cell Factories 2011, 10:61-78.
Talarico, T. L., Casas, I. A. Chung, T. C. and Dobrogosz, W. J. (1988) Production and Isolation of Reuterin, a Growth Inhibitor Produced by Lactobacillus reuterii. Antimicrobial Agents and Chemotherapy, 32: 1854-1858.
Vaidyanathan, H., Kandasamy, V., Ramakrishnan, G. G., Ramachandran, K. B., Guhan Jayaraman, G. and Ramalingam, S. (2014) Glycerol conversion to 1, 3-Propanediol is enhanced by the expression of a heterologous alcohol dehydrogenase gene in Lactobacillus Reuteri. AMB Express: 1:37-44.
Vollenweider, S. and C. Lacroix, C. (2004) 3-Hydroxypropionaldehyde: applications and perspectives of biotechnological production. Appl. Microbiol. Biotechno. 64:16-27.
Wang, Q., Ingram, L. O. and K. T. Shanmugam, K. T. (2011) Evolution of D-lactate dehydrogenase activity from glycerol dehydrogenase and its utility for D-lactate production from lignocellulose, Proc. Natl. Aca. Sci. USA 108:18920-18925.
Zeng, A-P. (2019) New bioproduction systems for chemicals and fuels: Needs and new development. Biotech, Advan. 37:508-518.

1-41. (canceled)

42. A process for recovering bioacrolein from a fermentation broth comprising:
(a) selecting a fermentation broth comprising bio-3-hydroxypropionaldehye;
(b) allowing the bio-3-hydroxy propionaldehyde to undergo a spontaneous dehydration reaction to yield acrolein; and
(c) recovering acrolein formed in step (b) by fractional distillation process.

43. The process for recovering bioacrolein according to claim 42, wherein the fermentation broth is associated with an ongoing biological fermentation.

44. The process for recovering bioacrolein according to claim 43, wherein the ongoing biological fermentation uses glycerol as a feedstock.

45. The process for recovering bioacrolein according to claim 42, wherein the bioacrolein recovered using distillation process is in the form acrolein azeotrope with water.

46. The process for recovering bioacrolein according to claim 45, wherein the acrolein azeotrope has 2-3% water content.

47. The process for recovering bioacrolein according to claim 42, wherein the fractional distillation process is carried out at acrolein azeotrope boiling point of 52.4° C., at a temperature higher than about 52.4° C. temperature, or at a temperature higher than the acrolein azeotrope boiling point of 52.4° C.

48. The process for recovering bioacrolein according to claim 42, wherein the fractional distillation process is carried out at a temperature lower than the acrolein azeotrope boiling point of 52.4° C.

49. The process for recovering bioacrolein according to claim 42, wherein the fractional distillation process is carried out at a vapor pressure lower than the 1,013.25 millibars.

50. The process for recovering bioacrolein according to claim 42, wherein the fractional distillation process is carried out at a vapor pressure lower than 1,013.25 millibars and at a temperature lower than the acrolein azeotrope boiling point of 52.4° C.

51. A method for producing bioacrolein comprising:
a) selecting a microorganism expressing glycerol dehydratase enzyme;
b) feeding glycerol to the microorganism in a) to produce bio-3-hydroxy propionaldehyde;
c) allowing the bio3-hydroxy propionaldehyde to undergo a spontaneous dehydration reaction to yield bioacrolein; and
d) recovering the bioacrolein formed in c) using a fractional distillation process.

52. The method for producing bioacrolein according to claim 51, wherein said microorganism contains an endogenous gene coding for glycerol dehydratase enzyme.

53. The method for producing bioacrolein according to claim 51, wherein said microorganism contains an exogenous gene coding for glycerol dehydratase enzyme.

54. The method for producing bioacrolein according to claim 53, wherein said exogenous glycerol dehydratase enzyme is a coenzyme-B12-dependent enzyme.

55. The method for producing bioacrolein according to claim 54, wherein said microorganism further comprises one or more exogenous genes coding for enzymes functional in B12 coenzyme biosynthesis.

56. The method for producing bioacrolein according to claim 53, wherein said exogenous glycerol dehydratase enzyme is a coenzyme-B12-independent enzyme.

57. The method for producing bioacrolein according to claim 51, wherein said microorganism expressing glycerol dehydratase is an acidophilic organism or is a thermophilic organism.

58. The method for producing bioacrolein according to claim 51, wherein said microorganism expressing glycerol dehydratase further comprises:
a) a mutation in a gene coding for NAD-linked glycerol dehydrogenase;
b) a mutation in a gene coding for dihydroxyacetone kinase;
c) a mutation in a gene coding for NADH-dependent oxidoreductase; or
d) a mutation in a gene coding for aldehyde dehydrogenase.

59. A microorganism comprising an exogenous gene coding for a glycerol dehydratase enzyme, wherein:
a) the microorganism is an acidophile; or
b) the microorganism is a thermophile.

60. A method of preparing bioacrylic acid comprising:
(a) preparing bioacrolein according to claim 51;
(b) oxidizing bioacrolein in step (a) to bioacrylic acid using chemical catalyst; and
(c) recovering bioacrylic acid.

61. The method of preparing bioacrylic acid according to claim 60, wherein the oxidation of bioacrolein is performed using a chemical catalyst.
